(19) United States
(12) Patent Application Publication
(10) Pub. No.: US 2014/0301937 A1
(43) Pub. Date: Oct. 9, 2014

(54) FULLERENE SEPARATION THROUGH USE OF ORGANIC CAGES

(71) Applicant: The Regents Of The University Of Colorado, a body corporate, Denver, CO (US)

(72) Inventors: Wei Zhang, Superior, CO (US); Qi Wang, Boulder, CO (US); Chenxi Zhang, Boulder, CO (US)

(21) Appl. No.: 14/354,479

(22) PCT Filed: Oct. 26, 2012
(86) PCT No.: PCT/US12/62080

§ 371 Date: Apr. 25, 2014

Related U.S. Application Data
(60) Provisional application No. 61/551,753, filed on Oct. 26, 2011.

(51) Int. Cl. C07D 487/00; C01B 31/02

(52) U.S. Cl. CPC C07D 487/00; C01B 31/0213
USPC 423/461; 540/145

(57) ABSTRACT

Provided herein are compositions useful in the separation of fullerenes from a mixture comprising fullerenes. Also provided herein are methods of making the compositions, as well as methods of using the compositions for fullerene separation.

### RELATED APPLICATION

This application claims the benefit U.S. Provisional application 61/551,753, filed on Oct. 26, 2011, the contents of which are incorporated herein in its entirety.

### GOVERNMENT SUPPORT

This invention was made with government support under grant numbers CBET 1033255 and DMR-1055705 awarded by the National Science Foundation. The government has certain rights in the invention.

### BACKGROUND OF THE INVENTION

Fullerenes are one of the four types of naturally occurring forms of carbon. They are distinguished by their multi-faceted, closed structure, where the carbon-carbon bonds form a framework of hexagons and pentagons that resembles the familiar hexagon/pentagon surface of a soccer ball. In general, more than one arrangement of the hexagons and pentagons is possible, leading to a great variety of possible isomers for any particular number of carbon atoms in a fullerene. One of the most common fullerenes is C₆₀, also referred to as Buckminsterfullerene, the structure of which is a network of hexagons and pentagons resembling a round soccer ball (Kroto, H. W. et al., “C₆₀: Buckminsterfullerene”, 318 Nature, pp. 162-163, November 1985). Other higher fullerenes such as C₇₀ have also been discovered.

Since the discovery of C₆₀, various potential applications of fullerenes have been identified, including using fullerenes as lubricants, controlled-release agent in drugs, and a component in superconductors. Other applications of fullerenes include optical devices, carbides, chemical sensors, gas separation devices, thermal insulation, diamonds, diamond thin films, and hydrogen storage. For example, [n]PCBM (phenyl Cₙ butyric acid methyl ester) fullerenes are used extensively in photovoltaics and polymer electronics.

The difficulties in the preparation, isolation and purification of fullerenes have greatly hindered their commercial exploitation. In particular, due to the highly similar structure, solubility, and reactivity of the fullerenes in a reaction mixture, with the various fullerenes only being differentiated in their molecular weight, it has been difficult to separate the discrete fullerene components from a crude fullerene mixture.

### SUMMARY OF THE INVENTION

Provided herein are compositions useful for the separation of fullerenes from any mixture comprising fullerenes. Specifically, alkyne metathesis has been used to construct the 3-D cubic molecular cages of Formula A (e.g., Formula I, e.g., COP-5) and Formula B (e.g., Formula II, e.g., Macrocycle 1), in one step from readily accessible precursors. Compounds of the Formula A consist of rigid, aromatic and carbazole moieties as well as linear ethynylene linkers, rendering its shape-persistent nature. In contrast, compounds of the Formula B are conformationally flexible even though they consist of highly rigid aromatic building blocks.

Accordingly, in one aspect, provided herein is a compound of the Formula A:

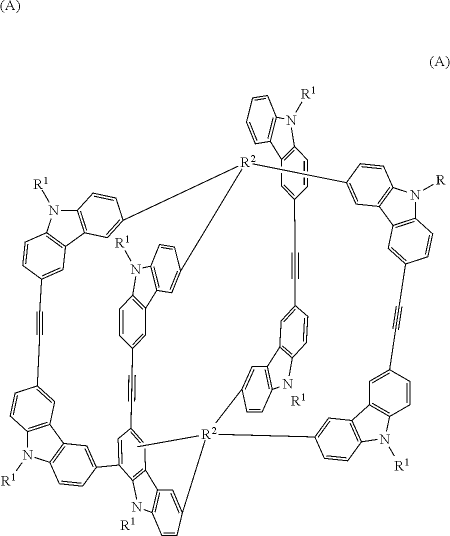

wherein

R¹ is a hydrophobic moiety or a hydrophilic moiety, and

R² is a monocyclic or fused hydrocarbon aromatic or heteroaromatic moiety, wherein the heteroaromatic moiety comprises one or more oxygen, nitrogen or phosphorous atoms.

In another aspect, provided herein is a compound of the Formula B:

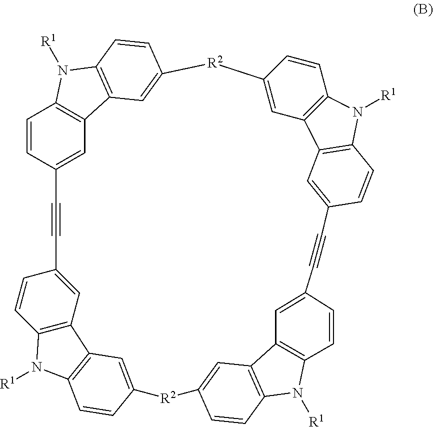

wherein

R¹ is a hydrophobic moiety or a hydrophilic moiety, and

R² is a monocyclic or fused hydrocarbon aromatic or heteroaromatic moiety, wherein the heteroaromatic moiety comprises one or more oxygen, nitrogen or phosphorous atoms, wherein R² is optionally further substituted with an aromatic group, and wherein the aromatic group is optionally further substituted with a C₁-C₆-alkyl. In an embodiment, R¹ is C₁-C₃₀-alkyl. In another embodiment, R¹ is polyethylene glycol (PEG).

In certain embodiments of Formula A and Formula B, R² is pyrene, porphyrin, or phthalocyanine. In still another embodiment of Formula A and Formula B, R² is porphyrin.

In an embodiment of Formula B, R² is optionally further substituted with a phenyl group, wherein the phenyl group is optionally further substituted with a C₁-C₆-alkyl.

In a particular embodiment, the compound of Formula A is a compound having the Formula I:

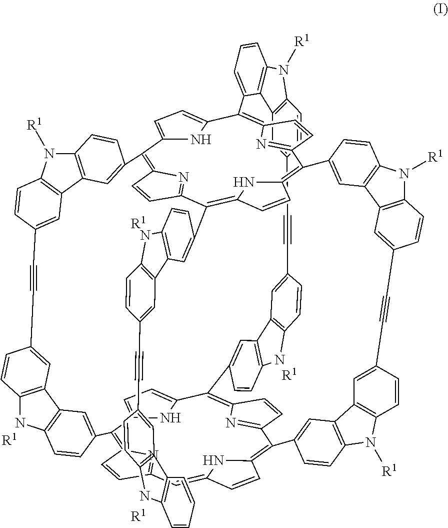

wherein R¹ is C₁-C₃₀-alkyl.

In another embodiment, the compound of Formula B is a compound having the Formula II:

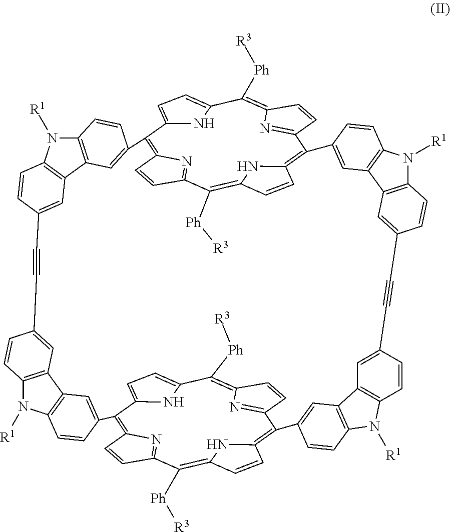

wherein R¹ is C₁-C₃₀-alkyl and R³ is C₁-C₆-alkyl.

In certain embodiments of Formula I and II, R¹ is C₁₀-C₂₀-alkyl. In another embodiment, R¹ is C₁₆H₃₃. In another embodiment, R³ of Formula II is t-butyl. In another embodiment, R³ is 4-t-butyl.

Also provided herein are methods of producing these compounds. In one aspect, provided herein is a method of preparing a compound of Formula I, comprising reacting a compound of Formula 3:

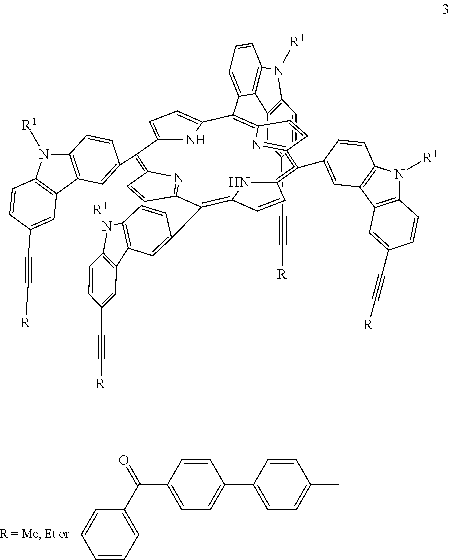

with a compound of formula 4:

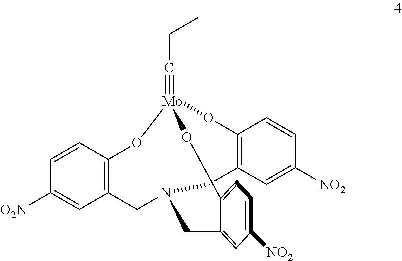

such that the compound of Formula I is produced, wherein R¹ is C₁-C₃₀-alkyl.

In another embodiment, provided herein is a method of preparing a compound of Formula II, comprising reacting a compound of Formula 5:

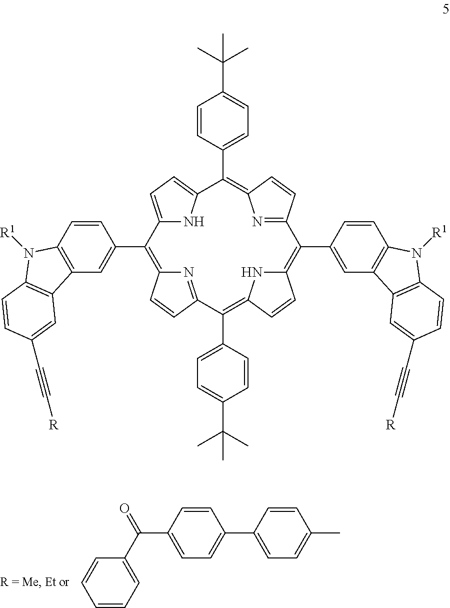

with a compound of formula 4:

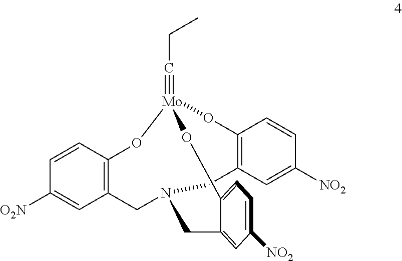

such that the compound of Formula II is produced, wherein R¹ is C₁-C₃₀-alkyl.

As described herein, the compounds of Formula I and II can serve as host molecules for fullerenes. Accordingly, in one aspect, provided herein is a method for separating fullerenes from a mixture comprising fullerenes, the method comprising contacting the mixture with a compound of Formula I to generate a Formula I-fullerene complex. Also provided herein is a method for separating fullerenes from a mixture comprising fullerenes, the method comprising contacting the mixture with a compound of Formula II to generate a Formula II-fullerene complex.

In one embodiment, the method for separating fullerenes using a compound of Formula I further comprises removing the Formula I-fullerene complex from the mixture. In an embodiment, the method further comprises separating the fullerene from the Formula I-fullerene complex. In an embodiment, the fullerene is separated from the Formula I-fullerene complex by contacting the complex with acid, for example, trifluroacetic acid.

In another embodiment, the method for separating fullerenes using a compound of Formula II further comprises removing the Formula II-fullerene complex from the mixture. In an embodiment, the method further comprises separating the fullerene from the Formula II-fullerene complex. In an embodiment, the fullerene is separated from the Formula II-fullerene complex by contacting the complex with acid, for example, trifluroacetic acid.

In an embodiment of these methods, the fullerene to be extracted is C₆₀, C₇₀, or a mixture thereof. In another embodiment, the fullerene to be extracted is C₈₄.

In an embodiment of the separation, the mixture containing fullerenes comprises at least one of C₆₀, C₇₀, C₇₆, or C₈₄, or other higher or lower molecular weight fullerenes represented by C₂₀₊₂ₘ where m is an integer.

In an aspect, provided herein is a method for separating C₇₀ fullerenes from a mixture comprising C₆₀ and C₇₀ fullerenes, wherein the method comprises contacting the mixture with a compound of Formula I. In an embodiment, the method further comprises removing the Formula I-C₇₀ complex from the mixture. The method can further comprise separating the C₇₀-fullerene from the Formula I-C₇₀ complex. In an embodiment, the C₇₀-fullerene is separated from the Formula I-C₇₀ complex by contacting the complex with acid. In one embodiment, the acid is trifluroacetic acid.

In another aspect, provided herein is a method for separating C₈₄ fullerenes from a mixture comprising C₈₄ fullerenes and at least one of C₆₀ or C₇₀ fullerenes, wherein the method comprises contacting the mixture with a compound of Formula II.

In certain embodiments of these methods, the separation takes place in a solvent. Non-limiting examples of solvents are tetrahydrofuran, dioxane, toluene, or dichloromethane.

In one aspect, provided herein is a complex comprising a compound of Formula I and C₇₀ fullerene. In another aspect, provided herein is a complex comprising a compound of Formula I and C₆₀ fullerene. In still another aspect, provided herein is a complex comprising a compound of Formula II and C₈₄ fullerene. In certain embodiments of these complexes, R¹ of Formula I or Formula II is C₁₀-C₂₀-alkyl. In still another embodiment, R¹ of Formula I or Formula II is C₁₆H₃₃.

In another aspect, provided herein is a molecular cage prepared from a single monomer, comprising the same top and bottom molecular structures, wherein the top and bottom molecules are linked through an ethynylene group to form a non-collapsible structure. In an embodiment, the top and bottom molecules are porphyrin or phthalocyanine. In another embodiment, the porphyrin or phthalocyanine groups are substituted with carbazole.

### BRIEF DESCRIPTION OF THE FIGURES

FIG. 1 shows energy-minimized structures of COP-5 (a, top view; b, side view), C₇₀@COP-5 (c), and C₆₀@COP-5 (d).

FIGS. 2a, 2b, and 2c show COP-5-fullerenes (C₇₀ and C₆₀) binding studies.

FIG. 3 shows ¹H NMR spectra of COP-5 and COP-5-fullerene complexes in C₆D₆: a) COP-5; b) C₆₀ @ COP-5; c) C₇₀@COP-5; d) a mixture of COP-5 with 10.0 equiv. of C₆₀ and 1.0 equiv. of C₇₀. C₆₀ @ COP-5 and C₇₀@COP-5 were prepared using 2 equiv. C₆₀ and 2 equiv. C₇₀ respectively.

FIG. 4 shows UV-Vis titration of macrocycle 1 with C₆₀(a), C₇₀(b), and C₈₄(c).

FIG. 5 is a schematic presentation of the C₇₀ isolation process.

FIGS. 6a and 6b demonstrate pH-driven reversible COP-5-fullerene binding.

FIG. 7 shows a synthesis procedure for the compound COP-5.

FIG. 8 shows the synthesis procedure for the compound macrocycle 1.

### DETAILED DESCRIPTION OF THE INVENTION

Three-dimensional (3-D) molecular cages, particularly shape-persistent, covalent organic polyhedrons (COPs) with well-defined pore dimensions have attracted considerable attention due to their numerous applications in host-guest chemistry, chemical sensing, catalysis, and gas adsorption. Current synthesis of rigid molecular cages is dominated by supramolecular chemistry including metal coordination (see, e.g., Olenyuk, B. et al., Nature 1999, 398, 796-799; Seidel, S. R. et al., J. Acc. Chem. Res. 2002, 35, 972-983; and Fiedler, D. et al., Acc. Chem. Res. 2005, 38, 349-358) and hydrogen-bonding (see, e.g., Liu, Y. Z. et al., Science 2011, 333, 436-440), which usually provides the target species with high efficiency through the self-assembly process. However, the supramolecular cages usually tend to be labile, and are sensitive to external environmental factors such as pH, temperature, solvent, etc. While supramolecular cages have been extensively studied, purely organic covalent molecular cages are relatively rare and have only recently received increasing attention. Conventionally, COPs are constructed via irreversible chemical transformations, which usually require enormous synthetic and purification efforts with very low overall yields. In great contrast, recent advances in dynamic covalent chemistry (DCC) are offering convenient pathways to high-yielding synthesis of COPs. To date, imine condensation/metathesis is almost the only reversible DCC reaction that has been used in construction of 3-D molecular architectures (see, e.g., Liu, X. J. et al., Angew. Chem. Int. Ed. 2006, 45, 901-904; and Meyer, C. D., et al., Chem. Soc. Rev. 2007, 36, 1705-1723). However, the potential drawbacks of imine groups are their sensitivity to acidic conditions and water. Further hydride reduction of imines provides more robust, but also flexible amino groups, resulting in the loss of certain shape-persistency of target structures.

Fullerenes can be produced by a variety of techniques, including high temperature vaporization of graphite. Such techniques also produce what is known as “fullerene soot.” Fullerene soot obtained by vaporization methods, etc., contains a fullerene mixture having any two or more of C₆₀, C₇₀ and higher fullerenes having greater than 70 carbon atoms (e.g., C₇₆, C₇₈, C₈₂, C₈₄, C₉₀, C₉₆, C₁₂₀, etc.), as well as soot residue (e.g., phenanthrene, pyrene, benzo[b]fluorene, benzo[c]phenanthrene, benzo[a]anthracene, triphenylene, benzopyrene, carbon having a graphite structure, carbonaceous polymers such as carbon black, and/or polycyclic aromatic hydrocarbons such as acenaphthylene).

Provided herein are compositions and methods that are useful for removing fullerenes from a composition comprising fullerenes, such as fullerene-containing soot. Specifically, provided herein are cubic molecular cages of Formula A (e.g., Formula I, e.g., COP-5) and Formula B (e.g., Formula II, e.g., Macrocycle 1).

### Compounds of Formula A and Formula B

Provided herein is a 3-D cubic molecular cage having the Formula A:

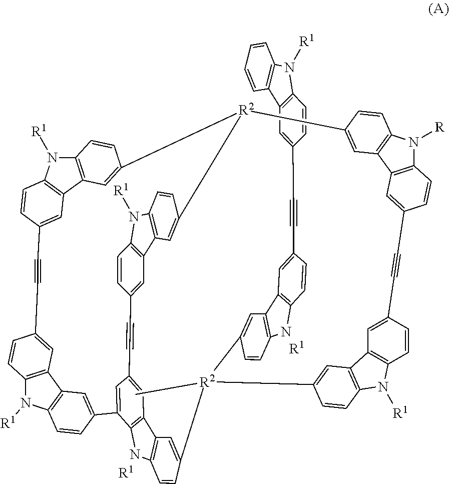

wherein R¹ is a hydrophobic moiety or a hydrophilic moiety, and R² is a monocyclic or fused hydrocarbon aromatic or heteroaromatic moiety, wherein the heteroaromatic moiety comprises one or more oxygen, nitrogen or phosphorous atoms.

In an embodiment of Formula A, R¹ is C₁-C₃₀-alkyl. In another embodiment of Formula A, R¹ is C₁₀-C₂₀-alkyl. In another embodiment, R¹ is C₁₆H₃₃. In another embodiment, R¹ is PEG.

In an embodiment of Formula A, R² is a monocyclic or fused hydrocarbon aromatic or heteroaromatic moiety comprising one or more oxygen, nitrogen or phosphorous atoms.

In one embodiment of Formula A, R² is pyrene, porphyrin, or phthalocyanine. In one embodiment of Formula A, R² is a porphyrin or phthalocyanine. In another embodiment, R² is porphyrin.

In one embodiment, the compound of Formula A has the Formula I:

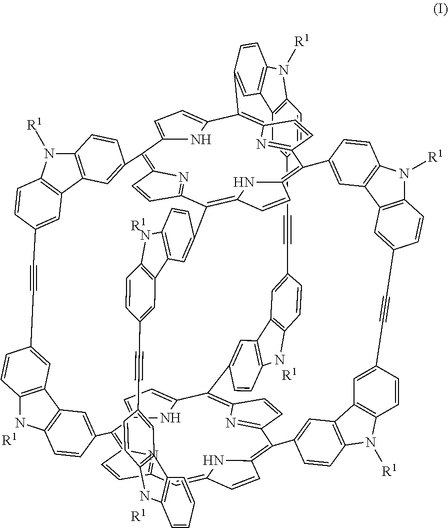

wherein R¹ is C₁-C₃₀-alkyl. In one embodiment of Formula I, R¹ is C₁₀-C₂₀-alkyl. In another embodiment, R¹ is C₁₆H₃₃ (also known as COP-5).

Also provided herein is a compound of the Formula B:

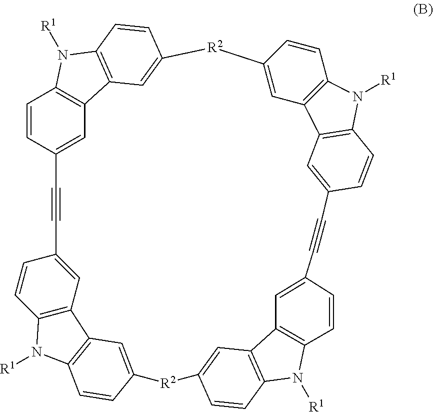

wherein R¹ is a hydrophobic moiety or a hydrophilic moiety, and R² is a monocyclic or fused hydrocarbon aromatic or heteroaromatic moiety, wherein the heteroaromatic moiety comprises one or more oxygen, nitrogen or phosphorous atoms, wherein R² is optionally further substituted with an aromatic group, and wherein the aromatic group is optionally further substituted with a C₁-C₆-alkyl.

In an embodiment of Formula B, R¹ is C₁-C₃₀-alkyl. In another embodiment of Formula B, R¹ is C₁₀-C₂₀-alkyl. In another embodiment, R¹ is C₁₆H₃₃. In another embodiment, R¹ is PEG.

In an embodiment of Formula B, R² is pyrene, porphyrin, or phthalocyanine. In another embodiment, R² is porphyrin.

In another embodiment of Formula B, R² is optionally further substituted with a phenyl group, wherein the phenyl group is optionally further substituted with a C₁-C₆-alkyl.

In one embodiment, Formula B is a compound having the Formula II:

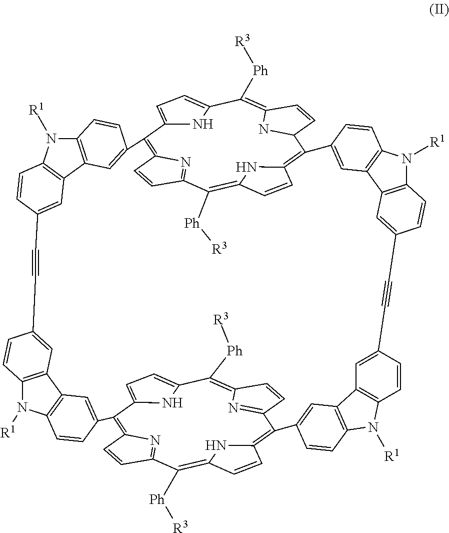

wherein R¹ is C₁-C₃₀-alkyl and R³ is C₁-C₆-alkyl. In an embodiment of Formula II, R¹ is C₁₀-C₂₀-alkyl. In another embodiment, R¹ is C₁₆H₃₃. In still another embodiment, R³ is t-butyl. In a particular embodiment of Formula II, R¹ is C₁₆H₃₃ and R³ is para-t-butyl (also known as macrocycle 1).

For compounds of Formula A and B, the term “hydrophobic moiety” refers to a moiety which itself is not wetted by water. Non-limiting examples of hydrophobic moieties include alkyl, alkenyl, alkynyl, cycloalkyl, haloalkyl, alkoxy, alkoxyalkyl, aryloxy, cycloalkoxy, alkylthio, alkanoyl, aroyl, substituted aminocarbonyl, and aminoalkanoyl, wherein these moieties have at least some hydrophobicity and generally have the properties of poor miscibility with water and low polarity.

For compounds of Formula A and B, a “hydrophilic moiety” is a moiety that exhibits characteristics of water solubility. In certain embodiments, the hydrophilic group is linear or a branched polymer or copolymer. Non-limiting examples of hydrophilic groups are: poly(ethylene glycol), alkoxy poly(ethyleneglycol), methoxy poly(ethylene glycol), dicarboxylic acid esterified poly(ethylene glycol) monoester, poly(ethylene glycol)-diacid, poly(ethylene glycol) monoamine, methoxy poly(ethylene glycol) monoamine, methoxy poly(ethylene glycol) hydrazide, methoxy poly(ethylene glycol) imidazolide, and poly-lactide-glycolide co-polymer.

For compounds of Formula A and B, the phrase “monocyclic or fused hydrocarbon aromatic” includes aromatic monocyclic or multicyclic e.g., tricyclic, bicyclic, or more, hydrocarbon ring systems consisting only of hydrogen and carbon and containing from six to 50 carbon atoms. The ring systems can be partially saturated. Aromatic groups can also be fused or bridged with alicyclic or heterocyclic rings which are not aromatic so as to form a polycycle (e.g., tetralin). Aromatic groups include, but are not limited to, those provided below in List 1:

### List 1

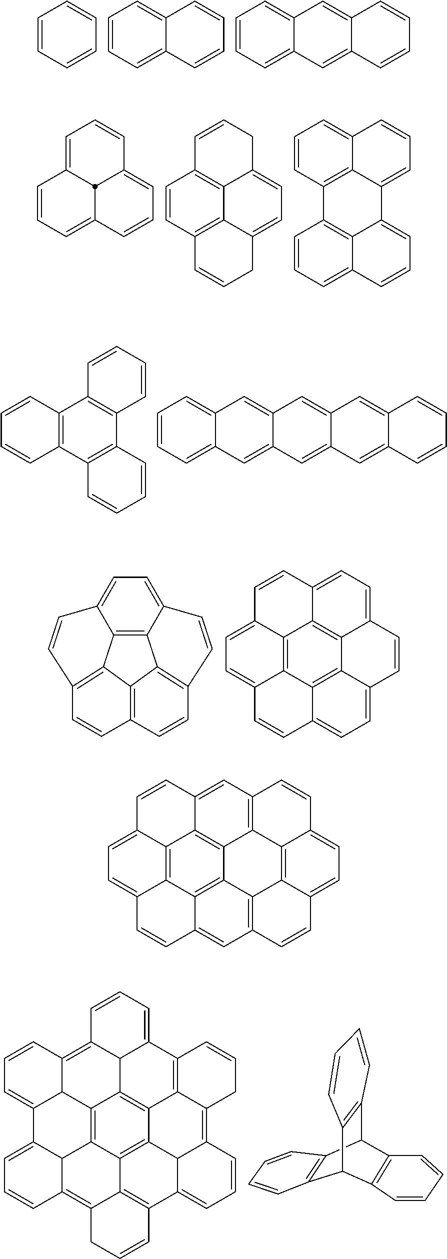

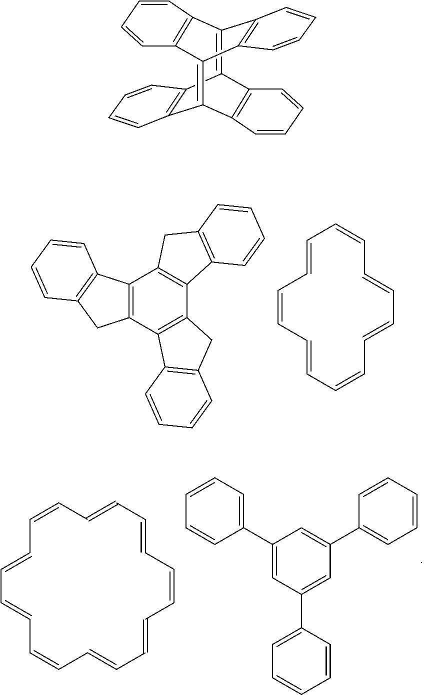

For compounds of Formula A and B, the phrase “monocyclic or fused heteroaromatic” represents a stable monocyclic or multicyclic ring system of up to 50 atoms, wherein at least one ring is aromatic and contains from 1 to 4 heteroatoms selected from the group consisting of O, N and S. Heteroaryl groups within the scope of this definition include but are not limited to, those provided below in List 2:

### List 2

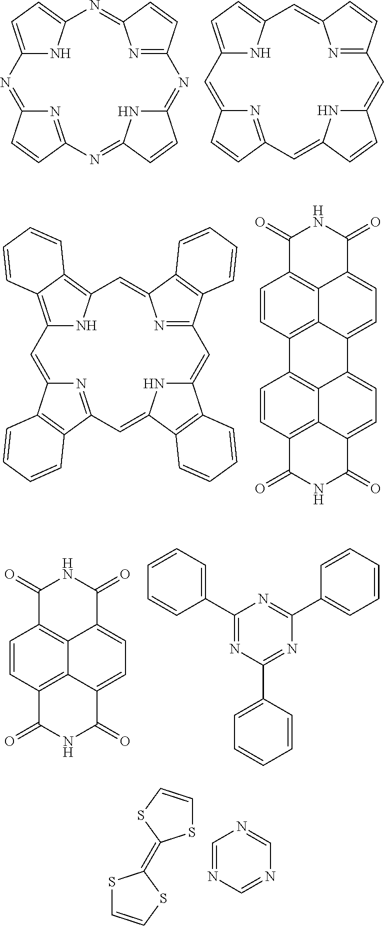

Compounds of Formula A (e.g., Formula I, e.g. COP-5), and compounds of Formula B (e.g., Formula II, e.g., Macrocycle 1) serve as an excellent receptor for fullerenes. For example, COP-5 forms 1:1 complexes with C₆₀ and C₇₀ with the association constants of 1.4×10⁵ M⁻¹ (C₆₀) and 1.5×10⁸ M⁻¹ (C₇₀) in toluene. This compound shows an unprecedented high selectivity in binding C₇₀ over C₆₀ (K_{C70}/K_{C60}>1000). Further, macrocycle 1 shows a strong binding interaction with fullerenes. In particular, this compound exhibits a high binding affinity for C₈₄ Moreover, the binding between these compounds and fullerene is fully reversible under the acid-base stimuli through a “Selective Complexation-Decomplexation” strategy.

Accordingly, in one aspect, provided herein is a method for separating fullerenes from a mixture comprising fullerenes, the method comprising contacting the mixture with a compound of Formula A to generate a Formula A-fullerene complex. The separation method can further comprise removing the Formula A-fullerene complex from the mixture. Once the complex is removed, the fullerene can be separated from the Formula A-fullerene complex.

In another aspect, provided herein is a method for separating fullerenes from a mixture comprising fullerenes, the method comprising contacting the mixture with a compound of Formula I to generate a Formula I-fullerene complex. The separation method can further comprise removing the Formula I-fullerene complex from the mixture. Once the complex is removed, the fullerene can be separated from the Formula I-fullerene complex.

In certain embodiments, the fullerene to be extracted is C₆₀, C₇₀, or a mixture thereof. In another embodiment of this method, the fullerene to be extracted is C₇₀. In another embodiment, the mixture containing fullerenes comprises C₆₀, C₇₀, C₇₆, or C₈₄, or other higher or lower molecular weight fullerenes represented by C₂₀₊₂ₘ where m is an integer. The mixture containing fullerenes can further comprise fullerene soot, as well as any of the common components of fullerene soot described above.

In another aspect, provided herein is a method for separating C₇₀ fullerenes from a mixture comprising C₆₀ and C₇₀ fullerenes, wherein the method comprises contacting the mixture with a compound of Formula A to generate a Formula A-C₇₀ complex. As part of the separation process, the Formula A-C₇₀ complex can be removed from the mixture. Further, the C₇₀-fullerene can be removed from the Formula A-C₇₀ complex.

In another aspect, provided herein is a method for separating C₇₀ fullerenes from a mixture comprising C₆₀ and C₇₀ fullerenes, wherein the method comprises contacting the mixture with a compound of Formula I to generate a Formula I-C₇₀ complex. As part of the separation process, the Formula I-C₇₀ complex can be removed from the mixture. Further, the C₇₀-fullerene can be removed from the Formula I-C₇₀ complex.

The fullerene can be separated from the Formula A-fullerene (e.g., C₇₀) complex by contacting the complex with acid. The acid, for example, an organic acid such as acetic acid, trifluoroacetic acid, or methanesulfonic acid, or an inorganic acid such as sulfuric acid, hydrochloric acid, or phosphoric acid can be added to the complex, thereby separating the fullerene from the compound of Formula A (e.g., Formula I). In a particular embodiment, the acid is trifluroacetic acid.

In another aspect, provided herein is a method for separating fullerenes from a mixture comprising fullerenes, the method comprising contacting the mixture with a compound of Formula B to generate a Formula B-fullerene complex. The separation method can further comprise removing the Formula B-fullerene complex from the mixture. Once the complex is removed, the fullerene can be separated from the Formula B-fullerene complex.

In another aspect, provided herein is a method for separating fullerenes from a mixture comprising fullerenes, the method comprising contacting the mixture with a compound of Formula II to generate a Formula II-fullerene complex. The separation method can further comprise removing the Formula II-fullerene complex from the mixture. Once the complex is removed, the fullerene can be separated from the Formula II-fullerene complex.

In certain embodiments, the fullerene to be extracted by a compound of Formula B (e.g., Formula II) is C₆₀, C₇₀, or a mixture thereof. In another embodiment of this method, the fullerene to be extracted is C₇₀. In certain embodiments, the fullerene to be extracted by a compound of Formula B (e.g., Formula II) is C₈₄. In another embodiment, the mixture containing fullerenes comprises C₆₀, C₇₀, C₇₆, or C₈₄, or other higher or lower molecular weight fullerenes represented by C₂₀₊₂ₘ where m is an integer. The mixture containing fullerenes can further comprise fullerene soot, as well as any of the common components of fullerene soot described above.

In another aspect, provided herein is a method for separating C₈₄ fullerenes from a mixture comprising C₈₄ fullerenes and at least one of C₆₀ and C₇₀ fullerenes, wherein the method comprises contacting the mixture with a compound of Formula B to generate a Formula B-C₈₄ complex. As part of the separation process, the Formula B-C₈₄ complex can be removed from the mixture. Further, the C₈₄-fullerene can be removed from the Formula B-C₈₄ complex.

In another aspect, provided herein is a method for separating C₈₄ fullerenes from a mixture comprising C₈₄ fullerenes and at least one of C₆₀ and C₇₀ fullerenes, wherein the method comprises contacting the mixture with a compound of Formula II to generate a Formula II-C₈₄ complex. As part of the separation process, the Formula II-C₈₄ complex can be removed from the mixture. Further, the C₈₄-fullerene can be removed from the Formula II-C₈₄ complex.

The fullerene can be separated from the Formula B-fullerene (e.g., C₈₄) complex by contacting the complex with acid. The acid, for example, an organic acid such as acetic acid, trifluoroacetic acid, or methanesulfonic acid, or an inorganic acid such as sulfuric acid, hydrochloric acid, or phosphoric acid can be added to the complex, thereby separating the fullerene from the compound of Formula B (e.g., Formula II). In a particular embodiment, the acid is trifluroacetic acid.

Any of the above separation procedures can be performed in a solvent, for example, a solvent in which fullerenes are soluble, e.g., an aromatic hydrocarbon, an aliphatic hydrocarbon or a chlorinated hydrocarbon, which may be cyclic or acyclic, and one or more of these solvents may be used in combination at any ratio.

Examples of aromatic hydrocarbon solvents are any hydrocarbon compounds having at least one benzene nucleus in a molecule, e.g., an alkylbenzene such as benzene, toluene, xylene, ethylbenzene, n-propylbenzene, isopropylbenzene, n-butylbenzene, sec-butylbenzene, tert-butylbenzene, 1,2,3-trimethylbenzene, 1,2,4-trimethylbenzene, 1,3,5-trimethylbenzene, 1,2,3,4-tetramethylbenzene, 1,2,3,5-tetramethylbenzene, diethylbenzene, and cymene; an alkylnaphthalene such as 1-methylnaphthalene and 2-methylnaphthalene; and tetralin.

The aliphatic hydrocarbon solvent can be either cyclic or acyclic. The cycloaliphatic hydrocarbon includes monocyclic aliphatic hydrocarbons such as cyclopentane, cyclohexane, cycloheptane, cyclooctane, and derivatives thereof such as methylcyclopentane, ethylcyclopentane, methylcyclohexane, ethylcyclohexane, 1,2-dimethylcyclohexane, 1,3-dimethylcyclohexane, 1,4-dimethylcyclohexane, isopropylcyclohexane, n-propylcyclohexane, tert-butylcyclohexane, n-butylcyclohexane, isobutylcyclohexane, 1,2,4-trimethylcyclohexane, and 1,3,5-trimethylcyclohexane. The cycloaliphatic hydrocarbon further includes polycyclic aliphatic hydrocarbons such as decalin, and acyclic aliphatic hydrocarbons such as n-pentane, n-hexane, n-heptane, n-octane, isooctane, n-nonane, n-decane, n-dodecane, and n-tetradecane.

The chlorinated hydrocarbon solvents include solvents such as dichloromethane, chloroform, carbon tetrachloride, trichloroethylene, tetrachloroethylene, 1,2-dichloroethane, 1,1,2,2-tetrachloroethane, chlorobenzene, dichlorobenzene, and 1-chloronaphthalene.

A ketone having 6 or greater carbon atoms, an ester having 6 or greater carbon atoms, an ether having 6 or greater carbon atoms (e.g., carbon disulfide) may also be used as a solvent. In an embodiment, the solvent is tetrahydrofuran, dioxane, toluene, or dichloromethane.

The solvents described above may be used alone, or two or more of these solvents may be used in combination as a mixed solvent.

### Fullerene Separation Using the Compounds of Formula a and Formula B

The unique conjugated system of these molecules results in rapid and selective binding of C₈₄, C₇₀ and C₆₀. For example, compounds of Formula A exhibit a three orders of magnitude stronger binding interaction with C₇₀ compared to C₆₀. Also, Compounds of Formula B forms a stable complex with C₈₄. Moreover, the clean release of fullerenes (guest) and regeneration of the compounds of Formula A (e.g., Formula I) and Formula B (e.g., Formula II) (host) was realized by simply tuning the pH of the media. Such “Selective Complexation-Decomplexation” strategy has been successfully applied to the isolation of C₆₀, C₇₀, and C₈₄ from fullerene mixtures.

Interestingly, the compounds of Formula A, e.g., compounds of Formula I, and compounds of Formula B, e.g., compounds of Formula II, are comprised of the same “top” and “bottom” pieces (e.g., a porphyrin or phthalocyanine moiety substituted with carbazole, which is linked to an identical moiety through an ethynylene linker), and no second type of building blocks are needed to form the cage. Such compounds consisting of only a single type of building units can be distinguished from those 3D molecular cages comprising different “side” pieces in addition to “top” and “bottom” pieces, which are prepared using, for example, imine condensation reactions.

Accordingly, in one aspect, provided herein is a molecular cage comprising a top and bottom molecule, wherein the top and bottom molecules have the same structure, and wherein the top and bottom molecules are linked through an ethynylene group to form a non-collapsible structure. In another aspect, provided herein is a molecular cage prepared from a single monomer, comprising the same top and bottom molecular structures, wherein the top and bottom molecules are linked through an ethynylene group to form a non-collapsible structure. In one embodiment, the top and bottom molecules are porphyrin or phthalocyanine moieties. The porphyrin or phthalocyanine groups can be substituted with carbazole.

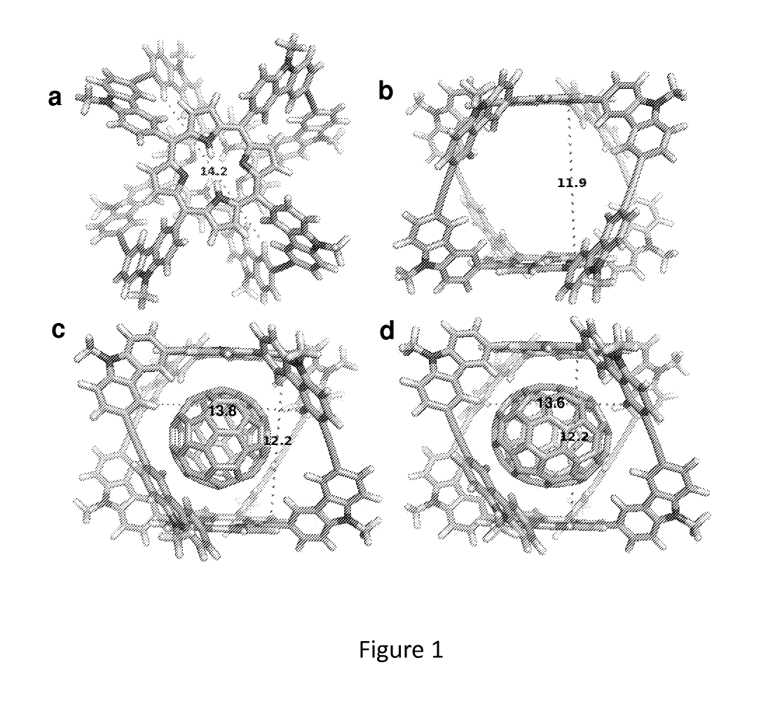

Porphyrin-fullerene binding is mainly driven by the electronic effect, i.e., the favored donor-acceptor interaction. Without being bound by theory, the computational modeling study (FIG. 1) reveals that COP-5 has a cavity with a height (defined as the distance between the top and bottom porphyrin panels) of 11.9 Å, and a diameter of 18.3 Å (defined as the distance between the two ethynylene groups in the diagonal edges), in which a fullerene can be nicely accommodated. FIG. 1 shows energy-minimized structures of COP-5 (a, top view; b, side view), C₇₀@COP-5 (c), and C₆₀@COP-5 (d). Methyl groups were used in the calculation instead of hexadecyl chains for simplification. The height of the COP-5 was defined as the distance between the top and bottom porphyrin panels, and the diameter of the inside cavity of COP-5 was defined as the distance between the two ethynylene groups in the diagonal edges.

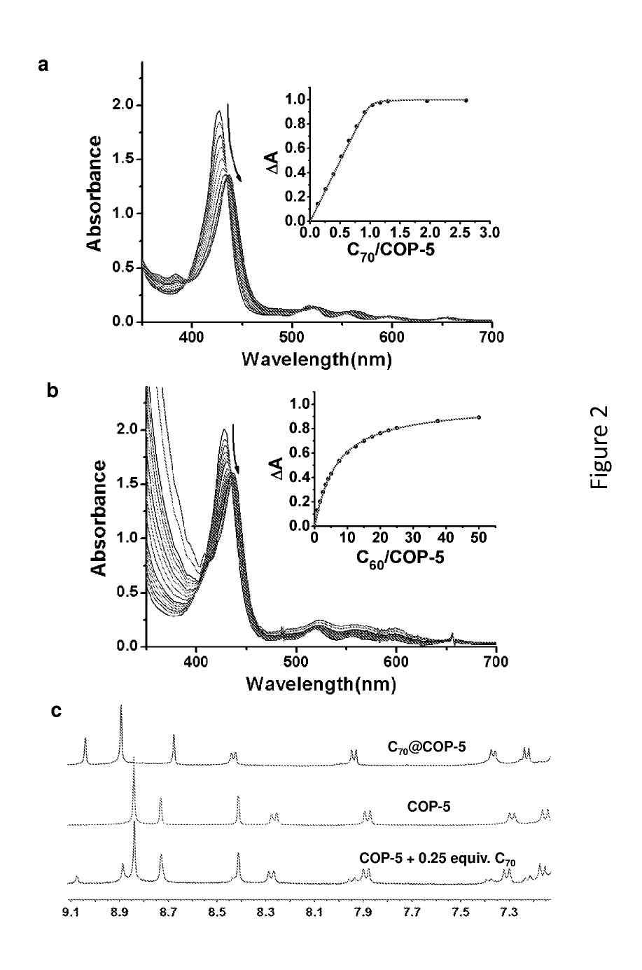

Compounds of Formula A, namely COP-5, showed a strong binding interaction with fullerenes. The binding of COP-5 with C₆₀ and C₇₀ was characterized by UV-Vis titration experiments in toluene (FIGS. 2a, 2b). With a gradual addition of C₇₀ to the cage solution (in toluene), the intensity of the absorption peak of COP-5 at 428 nm decreases while a new signal at 437 nm arises. The cage-C₇₀ complex (C₇₀@COP-5) formation is clearly signaled by the substantial intensity decrease (−55%, with 1 equiv. of C₇₀ added) at 428 nm and the red shift (9 nm) of the porphyrin Soret band compared to COP-5 itself. The similar trend in the UV-Vis titration curve was also observed when the solution of COP-5 in toluene was titrated with C₆₀. According to the Job plot, both C₆₀ and C₇₀ formed a 1:1 host-guest complex with COP-5 (data not shown). Consistent with this observation, when 0.25 eq. C₇₀ was added to the cage solution in toluene, the C₇₀@COP-5 was formed instantaneously, and the ¹H NMR spectra (FIG. 2c) showed two sets of signals corresponding to the free COP-5 and C₇₀@COP-5 complex with the integration ratio of 3:1, further supporting the 1:1 binding mode between COP-5 and C₇₀. MALDI-TOF spectra of cage-fullerene complexes clearly showed peaks with mass-to-charge ratio of 4544.20 and 4664.74 corresponding to 1:1 host-guest complex, C₆₀@COP-5 and C₇₀@COP-5, without any other complexes observed.

As summarized above, FIG. 2 shows COP-5-fullerenes (C₇₀ and C₆₀) binding studies. a, UV-Vis absorption spectra of COP-5 (2.0 μM) in toluene in the presence of various amounts of C₇₀ (0→3 equiv) at 23° C., while maintaining the concentration of COP-5 constant. Inset: plot of ΔA₄₂₈ nm vs. equivalents of C₇₀ added. b, UV-Vis absorption spectra of COP-5 (2.0 μM) in toluene in the presence of various amounts of C₆₀ (0→50 equiv) at 23° C., while maintaining the concentration of COP-5 constant. Inset: plot of ΔA₄₂₈ nm vs. equivalents of C₆₀ added. The association constants modeled with a 1:1 equilibrium, are K_{C70@COP-}5=1.5×10⁸ M⁻¹ (ΔG=−11.2 kcal/mol) for C₇₀@COP-5 and K_{C60@COP-}5=1.4×10⁵ M⁻¹ (ΔG=−7.0 kcal/mol) for C₆₀@COP-5. c, ¹H NMR spectra of C₇₀@COP-5 (blue), COP-5 (green), and a mixture of COP-5 with 0.25 eq. of C₇₀ (red) in toluene-d₈.

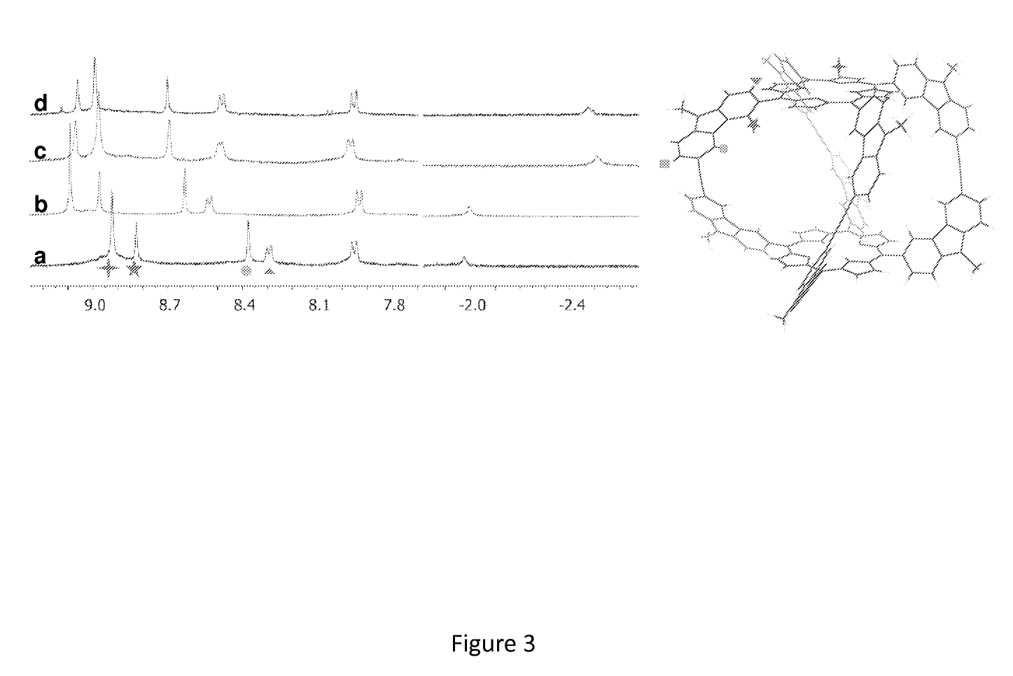

Additional evidence in support of the fullerene encapsulation inside the cage comes from the analysis of ¹H NMR spectra of the cage-fullerene complexes. The chemical shifts of the protons at the 4,5-positions on the carbazole corner pieces, which are pointing to the inside cavity of the cage, moved significantly downfield in both C₆₀@COP-5 and C₇₀@COP-5 while the other protons of the carbazole are not much affected (FIG. 3); such an observation indicates the fullerenes are located inside the cage. On the other hand, the internal N—H protons of the porphyrins at −1.97 ppm shifted significantly upfield by the influence of the fullerene ring current in C₇₀@COP-5 (FIG. 3), which indicates a strong π-π interaction between C₇₀ and the porphyrin panels of COP-5. The simplicity of the NMR signals of the C₇₀@COP-5 and C₆₀@COP-5 suggests a highly symmetrical structure of the complexes, further supporting the notion of fullerene binding inside the cubic cage.

Based on the 1:1 binding mode and fitting of the UV-Vis adsorption changes at 428 nm under different fullerene concentrations, the association constants of C₆₀ and C₇₀ with COP-5 were estimated to be 1.4×10⁵ M⁻¹ (C₆₀) and 1.5×10⁸ M⁻¹ (C₇₀) in toluene, which are comparable to those best performing fullerene receptors reported thus far.

It is noteworthy that the cubic cage COP-5 containing non-metallated porphyrin moieties shows a high binding affinity with C₇₀, which is three orders of magnitude higher than that with C₆₀. In order to further explore the potential of COP-5 in fullerene separation, a mixture of two fullerene guests was used in a binding competition test. As expected, selective complexation of COP-5 with C₇₀ in a C₆₀-enriched fullerene mixture was observed. Upon mixing COP-5 with a solution of C₆₀ (91 mol %) and C₇₀ (9 mol %) in toluene, the COP-5 selectively bound with C₇₀ to form C₇₀@COP-5. The ¹H NMR spectrum clearly shows the major set of proton signals corresponding to the C₇₀@COP-5 (FIG. 3).

Computational calculations on the energy-minimized structures of COP-5 and COP-5-fullerene complexes provide further insight into the preferential binding of C₇₀ versus C₆₀. The computational modeling study (FIG. 1) reveals that the heights of the COP-5 are slightly increased to 12.1-12.2 Å in C₆₀@COP-5 and C₇₀@COP-5 from initial 11.9 Å in the unoccupied COP-5, while the diameters of the cavities are decreased slightly to 17.6 Å-17.8 Å from 18.3 Å. Owing to the high degree of shape-persistency of cage COP-5, the size and geometry of the cage does not change much upon the fullerene encapsulation. In both C₆₀@COP-5 and C₇₀@COP-5 complexes, the shortest atom-to-atom distances between porphyrin panels and fullerenes are similar and close to 3.2 Å, which leads to an appreciable π-π interaction in both complexes. It is known that fullerenes also have CH-π it interactions with host molecules, and the favored distance for CH-π it interactions is around 2.9 Å (Nishio, M. et al. Tetrahedron 1999, 55, 10047-10056; Umezawa, Y. et al. Bull. Chem. Soc. Jpn. 1998, 71, 1207-1213). This type of CH-π interactions also exists in fullerene-COP-5 complexes. The average distances between fullerenes and those carbazole CH protons, which are pointing to the inside cavity of the cage, in C₆₀@COP-5 and C₇₀@COP-5 are around 3.5 Å and 3.1 Å respectively. Since the distance for CH-π it interactions in C₇₀@COP-5 (3.1 Å) is closer to the known favored distance 2.9 Å, the CH-π it interactions are presumably stronger in C₇₀@COP-5 compared to C₆₀@COP-5.

Accordingly, in one aspect, provided herein is a complex comprising a compound of Formula A and C₇₀ fullerene. In another aspect, provided herein is a complex comprising a compound of Formula A and C₆₀ fullerene. In either of these complexes, R¹ can be C₁₀-C₂₀-alkyl, e.g., C₁₆H₃₃ alkyl.

In another aspect, provided herein is a complex comprising a compound of Formula I and C₇₀ fullerene. In another aspect, provided herein is a complex comprising a compound of Formula I and C₆₀ fullerene. In either of these complexes, R¹ can be C₁₀-C₂₀-alkyl, e.g., C₁₆H₃₃ alkyl.

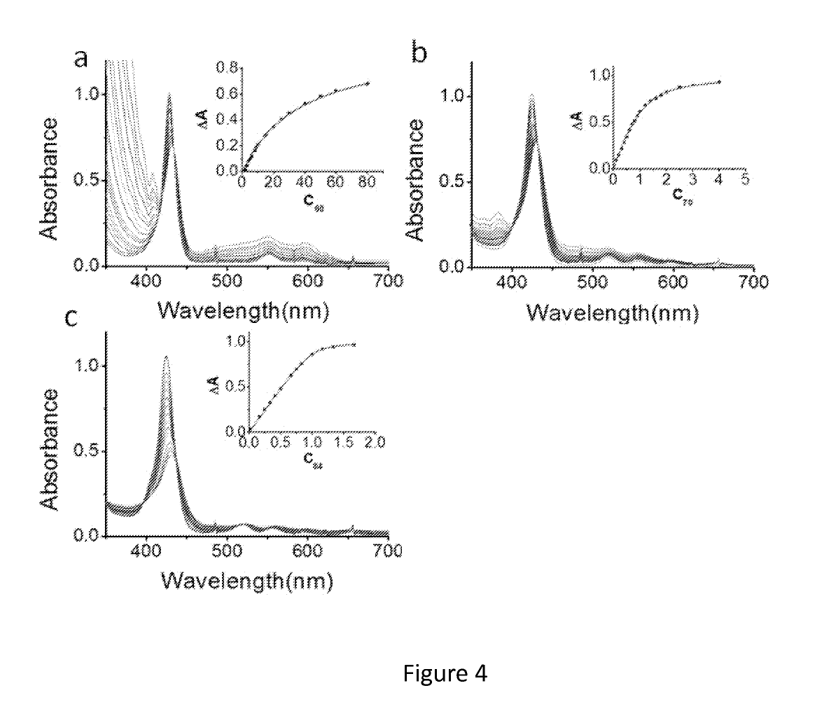

Similarly, compounds of Formula B, e.g., compounds of Fomrula II, e.g., macrocycle 1 showed a strong binding interaction with fullerenes. The binding of macrocycle 1 with C₆₀, C₇₀, and C₈₄ was characterized by UV-Vis titration experiments in toluene (FIG. 4a-c). In all cases, the decrease of the absorption peak at λₘₐₓ≈425 nm was observed, and the appearance of a new peak at λₘₐₓ≈430 nm was also observed. Macrocycle 1 forms a 1:1 host-guest complex with C₆₀, C₇₀, and C₈₄, respectively, based on the Job plot. The peaks corresponding to the complex C₇₀@1, and C₈₄@1 on the MALDI-TOF mass spectra of the cage-fullerene mixtures were also observed. The association constants of C₆₀, C₇₀ and C₈₄ with macrocycle 1 were calculated based on the 1:1 binding mode and fitting of the UV-Vis adsorption data at 425 nm under different fullerene concentrations. The results are summarized in Table 1.

TABLE 1
Binding constants of Macrocycle 1

| | K_{C60} (M⁻¹) | K_{C70}(M⁻¹) | K_{C84} (M⁻¹) |
|---|---|---|---|
| 1 | 1.3 × 10⁴ | 2.0 × 10⁶ | 2.2 × 10⁷ |

FIG. 4 shows the UV-Vis titration of macrocycle 1 with C₆₀(a), C₇₀(b), and C₈₄(c). The titration was conducted in toluene, the concentration of macrocycle 1 was 10⁻⁶ mol/L. Inset: plot of normalized ΔA_{425 nm} vs. equivalent of fullerene added. The absorbance at 425 nm was used for the titration of macrocycle 1.

Accordingly, in one aspect, provided herein is a complex comprising a compound of Formula B and C₈₄ fullerene. In another aspect, provided herein is a complex comprising a compound of Formula B and C₇₀ fullerene. In another aspect, provided herein is a complex comprising a compound of Formula B and C₆₀ fullerene. In either of these complexes, R¹ can be C₁₀-C₂₀-alkyl, e.g., C₁₆H₃₃ alkyl.

Accordingly, in one aspect, provided herein is a complex comprising a compound of Formula II and C₈₄ fullerene. In another aspect, provided herein is a complex comprising a compound of Formula II and C₇₀ fullerene. In another aspect, provided herein is a complex comprising a compound of Formula II and C₆₀ fullerene. In either of these complexes, R¹ can be C₁₀-C₂₀-alkyl, e.g., C₁₆H₃₃ alkyl.

### Fullerene Release

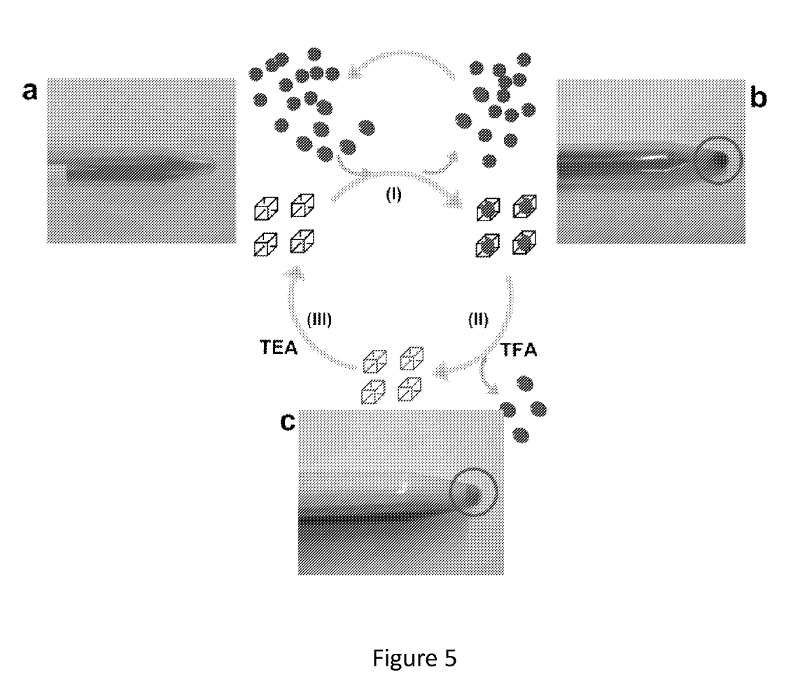

The difficult release of fullerenes and regeneration of hosts have greatly impeded the practical applications of host/guest chemistry (Komatsu, N. J. Inclusion Phenom. mol. Recognit. Chem. 2008, 61, 195-216). Given the over 1000 times stronger binding interactions of COP-5 with C₇₀ over C₆₀, and the reversible nature of this host-guest binding triggered by pH, C₇₀ from C₆₀ can be achieved with a simple “selective complexation-decomplexation” process (FIG. 5).

FIG. 5 is the schematic presentation of the C₇₀ isolation process. Step (I): To a solution of C₆₀ and C₇₀ mixture (C₆₀/C₇₀=10/1, molar ratio) was added a small amount of COP-5 (equal to or less than the stoichiometric amount of C₇₀), resulting in the favored formation of C₇₀@COP-5. After separating the unbound free fullerenes by precipitation in CHCl₃ (precipitates shown in b), cage-fullerene complexes (mostly C₇₀@COP-5) were collected in the solution phase; Step (II): Upon the addition of 100 equiv. TFA to the solution collected in the step II, fullerene guest molecules (mostly C₇₀) were released as black precipitates and removed to complete one cycle of the isolation process (shown in c); Step (III): Regeneration of COP-5 was accomplished by the addition of 100 equiv. TEA to the above solution.

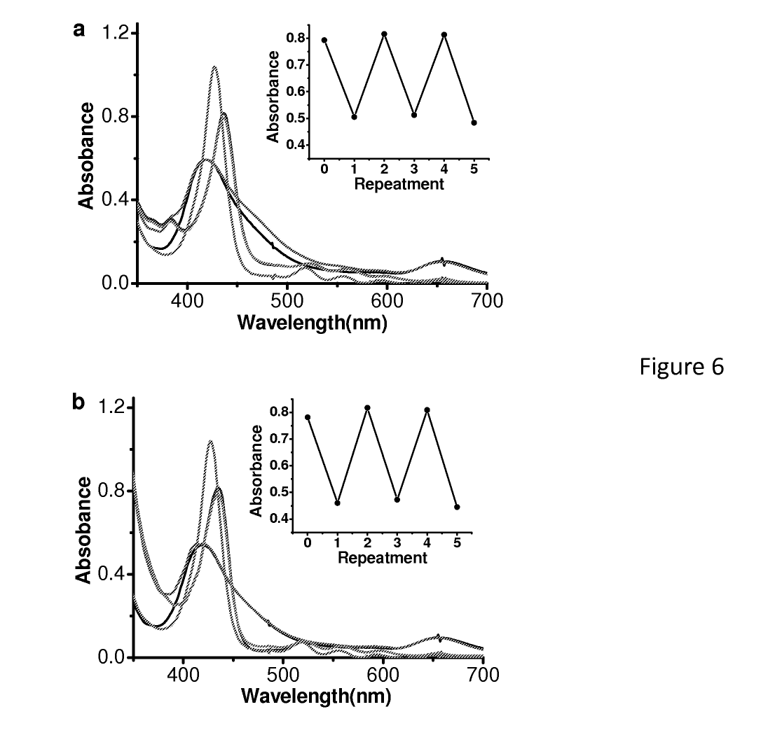

In an embodiment, the porphyrin-fullerene interactions can be tuned by changing the electronic properties of either one of them. Unlike metalloporphyrins, electron density of the porphyrin free base can be easily reduced by simple protonation, and thus the porphyrin-fullerene interactions could be weakened. Thus, for example, once a compound of Formula A (e.g., COP-5) is complexed with a fullerene (e.g., C₇₀ or C₆₀), that fullerene can be released through reaction with acid. For example, the dissociation and release of the guest molecules, and regeneration of the COP-5 or macrocycle 1 can be realized by simply tuning the pH of the media. Trifluoroacetic acid (TFA) and triethylamine (TEA) were used as the acid and base stimuli. Upon addition of excess TFA (100 equiv.) to the solution of C₇₀@COP-5 (or C₆₀@COP-5) in toluene, protonation of the porphyrin ring occurred, and consequently, complete release of the fullerene molecules was observed as evidenced by the disappearance of the ¹H NMR signals corresponding to C₇₀@COP-5 (or C₆₀@COP-5), and appearance of a new set of signals corresponding to the protonated COP-5 with an empty cavity. However, the subsequent addition of 100 equiv. triethylamine (TEA) to the above mixture neutralized the porphyrin ring and restored the binding interaction between COP-5 and fullerenes. As a result, the ¹H NMR spectrum of C₇₀@COP-5 (or C₆₀@COP-5) was regenerated, indicating the reversibility of the association/dissociation process. Such a reversible association/dissociation triggered by acid/base stimuli was also confirmed by monitoring the process with UV-Vis absorption spectra (FIG. 6). Almost the identical UV-Vis absorption spectra was observed when COP-5 and C₇₀@COP-5 (or C₆₀@COP-5) were protonated with 100 equiv. TFA, which indicates the complete dissociation of the cage-fullerene complex upon protonation of the porphyrin free base center. The regeneration of C₇₀@COP-5 (or C₆₀@COP-5) complex by the addition of 100 equiv. TEA to the mixture of protonated COP-5 and free C₇₀, was evidenced by the almost identical UV-Vis absorption as the pure C₇₀@COP-5 (or C₆₀@COP-5). The association/dissociation process can be repeated in several cycles without leading to any noticeable change in the absorbance by the alternating addition of TFA and TEA (FIG. 6, inset), thus showing the cage-C₇₀ binding is a highly efficient, robust and fully reversible process.

FIG. 6 demonstrates pH-driven reversible COP-5-fullerene binding. COP-5 concentration was 1.0×10⁻⁶M in toluene. a, UV-Vis spectrum of the free COP-5 (cyan); C₇₀@COP-5 (magenta); after the addition of 100 equiv. TFA to COP-5 (black); after the addition of 100 equiv. TFA to C₇₀@COP-5 (red), After the addition of 100 equiv. TFA, followed by the subsequent addition of 100 equiv. TEA to C₇₀@COP-5 (blue). Inset: Plot of absorption at 437 nm vs. repetitive association/dissociation cycles b, UV-Vis spectrum of the free COP-5 (cyan); C₆₀@COP-5 (magenta); after the addition of 100 equiv. TFA to COP-5 (black); after the addition of 100 equiv. TFA to C₆₀@COP-5 (red), After the addition of 100 equiv. TFA followed by the subsequent addition of 100 equiv. TEA to C₆₀@COP-5 (blue). Inset: Plot of absorption at 436 nm vs. repetitive association/dissociation cycles.

As a proof-of-concept, C₆₀-enriched C₆₀/C₇₀ mixture in the separation study was used. As discussed above, separation procedures can be performed in a solvent, for example, a solvent in which fullerenes are soluble, e.g., an aromatic hydrocarbon, an aliphatic hydrocarbon or a chlorinated hydrocarbon, which may be cyclic or acyclic, and one or more of these solvents may be used in combination at any ratio. In a non-limiting example, carbon disulfide was chosen as the solvent for the encapsulation step since both C₆₀ and C₇₀ have good solubility in CS₂. A mixture of COP-5, and C₆₀/C₇₀ in CS₂ was sonicated for 30 seconds and the solvent was evaporated. The residue was then dispersed in chloroform. Since fullerenes have very limited solubility in CHCl₃, free fullerenes remained as precipitates. The solution phase that is composed of mostly C₇₀@COP-5 was separated from the insoluble fullerene mixtures by centrifugation. Further acidifying the C₇₀@COP-5 complex with excess TFA followed by sonication (5 min) released C₇₀ as black precipitates, which allows easy removal by centrifugation. The cage COP-5 was then regenerated by neutralization with TEA and was recycled for the next round of fullerene separation. The UV-Vis absorption showed that the C₇₀ abundance of the fullerene mixture increased significantly (˜9 fold increase) from initial 9 mol % to 79 mol % after only one cycle of separation. This result clearly demonstrates the simplicity and high efficiency of such fullerene separation approach by using shape-persistent molecular cages as selective receptors. The “Selective Complexation-Decomplexation” strategy presented here will greatly facilitate the purification of these intriguing graphitic materials and promote their wide applications in organic photovoltaics, polymer electronics and biopharmaceuticals.

Given the high binding selectivity of macrocycle 1 toward C₈₄, the feasibility of the pH-controlled release of C₈₄ and regeneration of host macrocycle 1 was also explored. Addition of excess TFA (100 equiv.) to the solution of C₈₄@1 in toluene protonates the porphyrin ring and weakens the porphyrin-fullerene interaction, thus leading to the dissociation of C84 and macrocycle 1. As a consequence, a broadening and red shift of the adsorption band of C84@1 with the appearance of a new absorption band at 675 nm was observed. The absorbance of the acidified C₈₄@1 complex is in good agreement with the acidified 1 itself, which indicates C₈₄ released from the cage. Subsequent addition of triethylamine (100 equiv.) to the above mixture neutralized the porphyrin ring and restored the binding interaction between macrocycle 1 and C₈₄. Remarkably, the acid/base-mediated association/dissociation of the host-guest complex could be repeated many times without obvious change in the absorbance.

The over 1500 times stronger binding interaction of macrocycle 1 with C₈₄ over C₆₀, and the reversible nature of this host-guest binding triggered by pH open the possibility of using such “Selective Complexation-Decomplexation” approach for purification of higher fullerenes (e.g., C₈₄).

### Synthesis

In another aspect, provided herein is a method of preparing a compound of Formula I, comprising reacting a compound of Formula 3:

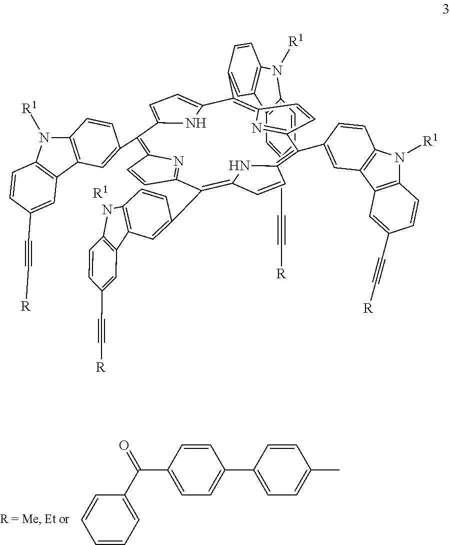

with a compound of formula 4:

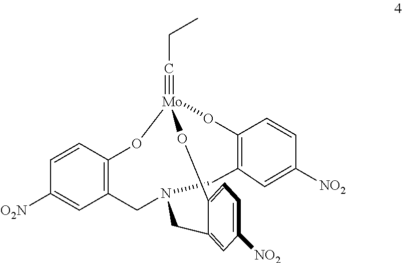

such that a compound of Formula I is produced, wherein R¹ is C₁-C₃₀-alkyl. In one embodiment, R¹ is C₁₀-C₂₀-alkyl. In another embodiment, R¹ is C₁₆H₃₃.

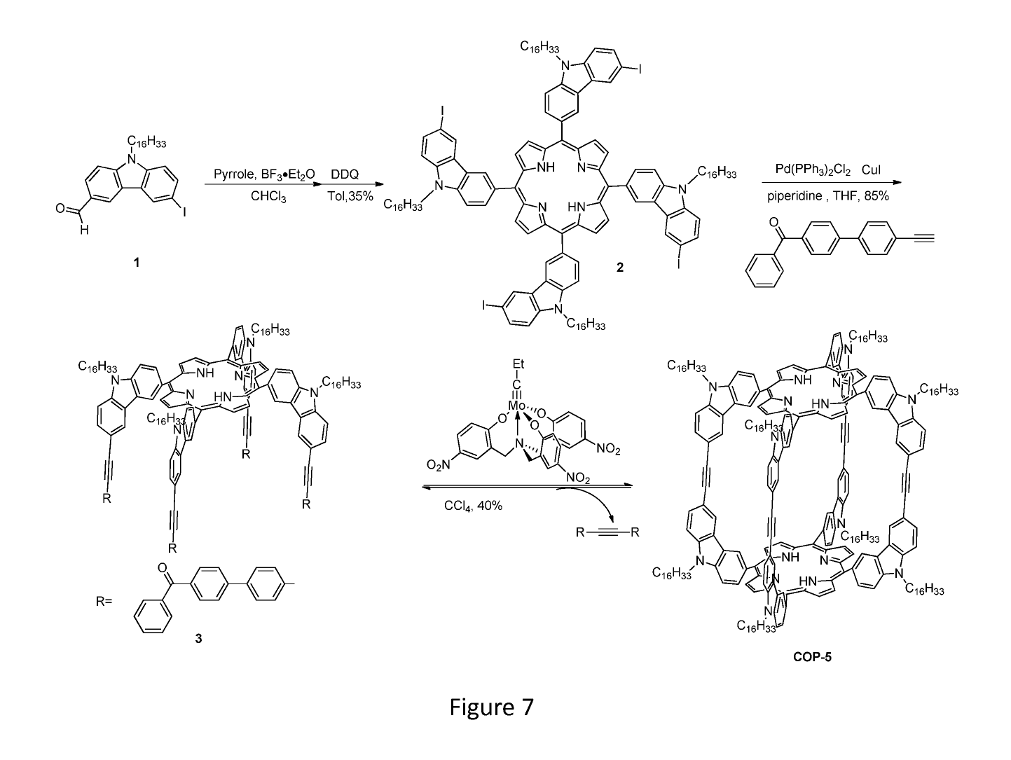

Scheme 1 of FIG. 7 shows a synthesis scheme for the preparation of a specific compound of the Formula A (e.g., Formula I). A brief description of this synthesis follows:

The monomer 3 was prepared from 3-iodo-6-formyl-9-hexadecylcarbazole 1 through Lindsey method to form 5,10,15,20-tetrakiscarbazolyl-porphyrin 2, followed by Sonogashira coupling to attach benzoylbiphenyl acetylene group. Benzoylbiphenyl was utilized as the end group so that insoluble byproduct diarylacetylenes would be formed along the reaction, thus driving the reversible alkyne metathesis to completion (Zhang, W. et al. J. Am. Chem. Soc. 2004, 126, 12796-12796). The reaction was performed at 75° C. under microwave irradiation in CCl₄. After 32 h, the predominant formation of cage COP-5 was observed (40% isolated yield). The cage formation via alkyne metathesis is a fully reversible process, which is evidenced by the gradual conversion of initial high molecular weight oligomeric intermediates into the final cubic cage COP-5 as shown in the reaction progress monitored by the gel permeation chromatography (GPC). Such a cubic cage is enthalpy-favored due to its minimal angle-strain and also entropy-favored due to its consisting minimal number of building blocks (compared to larger oligomeric products).

The molecular cube COP-5 was fully characterized by ¹H NMR, ¹³C NMR spectroscopy, UV-Vis spectroscopy, GPC, as well as MALDI-TOF mass spectrometry. The ¹H NMR spectrum of COP-5 in CDCl₃ shows only one set of singlet corresponding to the porphyrin protons at 8.73 ppm, indicating the high symmetry of the cage structure. The MALDI-TOF mass spectrum shows the desired molecular ion peaks at m/z 3825.80 ([M+H]⁺ calcd. for C₂₇₂H₃₃₂N₁₆: 3825.66), further confirming the formation of molecular cube COP-5. The cage is thermally stable and also exhibits a very high chemical stability even with exposure to water and acids (e.g., trifluoroacetic acid, TFA) for weeks, thus showing a great advantage over those supramolecular cages as well as imine-linked COPs.

In another aspect, provided herein is a method of preparing a compound of Formula II, comprising reacting a compound of Formula 5:

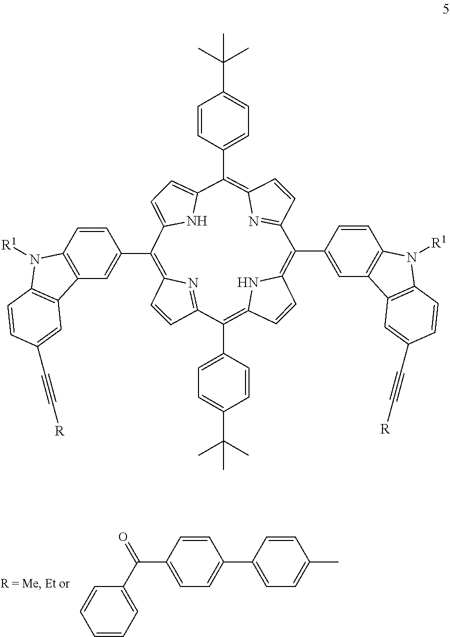

with a compound of formula 4:

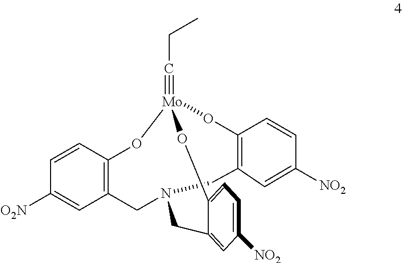

such that the compound of Formula II is produced, wherein R¹ is C₁-C₃₀-alkyl.

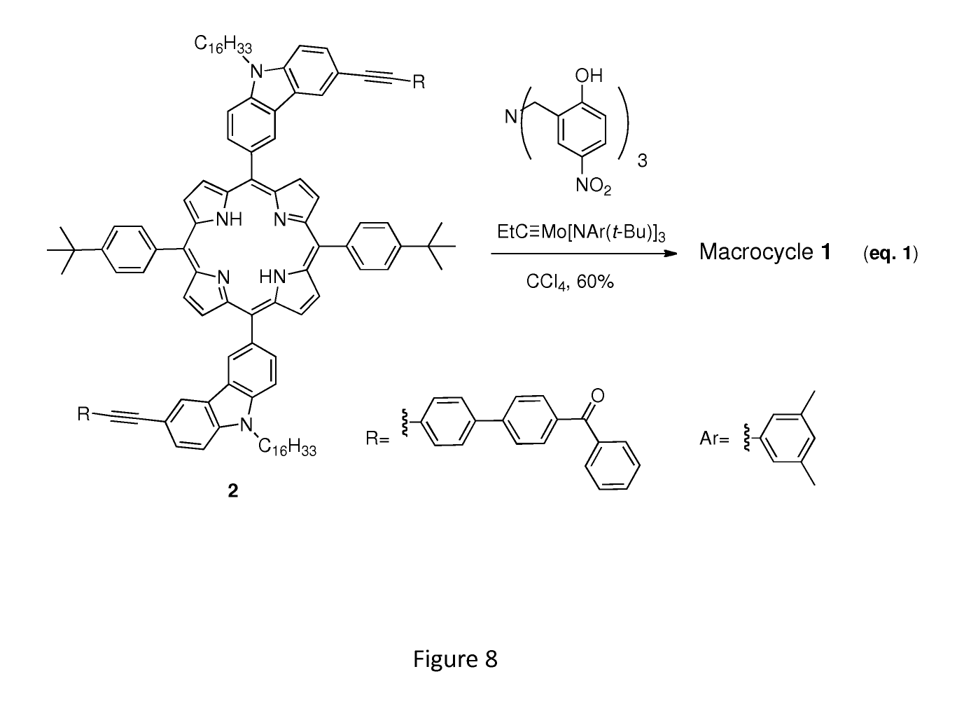

For example, as shown in FIG. 8, the bisporphyrin macrocycle 1 was prepared from porphyrin-based diyne monomer 2 through one-step alkyne metathesis (Eq. 1), catalyzed by a multidentate Mo(VI) alkylidyne catalyst. Porphyrin diyne 2 was synthesized from N-hexadecyl-3-formyl-6-iodocarbazole and 5-(4-tert-butylphenyl)-dipyrromethane through ring cyclization under the standard Lindsey conditions, followed by Sonogashira coupling reaction to install the end groups for precipitation-driven alkyne metathesis. The metathesis reaction was conducted at 45° C. for 16 hours to give the macrocycle 1 in 60% isolated yield. The gel permeation chromatography (GPC) trace of the crude reaction mixture showed the transformation of monomer 2 into the target macrocycle 1 without initial formation of a large amount of oligomers or polymers along the reaction process. Macrocycle 1 was purified by column chromatography, and characterized by ¹H, ¹³C NMR, MALDI-MS and GPC.

### Experimental

### Materials and General Synthetic Methods

Reagents and solvents were purchased from commercial suppliers and used without further purification, unless otherwise indicated. Tetrahydrofuran (THF), toluene, CH₂Cl₂ and dimethylformamide (DMF) are purified by the MBRAUN solvent purification systems.

All reactions were conducted under dry nitrogen in oven-dried glassware, unless otherwise specified. Solvents were evaporated using a rotary evaporator after workup. Unless otherwise specified, the purity of the compounds was ≧95% based on ¹H NMR spectral integration.

Flash column chromatography was performed by using a 100-150 times weight excess of flash silica gel 32-63 μm from Dynamic Absorbants Inc. Fractions were analyzed by TLC using TLC silica gel F254 250 μm precoated-plates from Dynamic Absorbants Inc. Analytical gel permeation chromatography (GPC) was performed using a Viscotek GPCmax™, a Viscotek Model 3580 Differential Refractive Index (RI) Detector, a Viscotek Model 3210 UV/VIS Detector and a set of two Viscotek Viscogel columns (7.8×30 cm, 1-MBLMW-3078, and 1-MBMMW-3078 columns) with THF as the eluent at 30° C. The analytical GPC was calibrated using monodisperse polystyrene standards.

UV-vis absorption measurements were carried out with Agilent 8453 spectrophotometer and the emission measurements were obtained on a F-2500 Hitachi fluorescence spectrophotometer.

MALDI Mass spectra were obtained on the Voyager-DE™ STR Biospectrometry Workstation using sinapic acid as the matrix. The high resolution Mass spectra were obtained on Waters SYNAPT G2 High Definition Mass Spectrometry System. Analyte molecules were diluted into ESI solvents, either methanol or acetonitrile/water mixture, for final concentrations of 10 ppm or lower. The solution was injected into the electrospray ionization (ESI) source at a rate of 5 μL/min. Either the ESI+ or ESI− mode was used in reference to the molecular properties. Accurate mass analysis was performed by using the Lock Mass calibration feature with the instrument.

NMR spectra were taken on Inova 400 and Inova 500 spectrometers. CHCl₃ (7.27 ppm), benzene-d₆ (7.15 ppm) and toluene-d₈ (2.09 ppm) were used as internal references in ¹H NMR, and CHCl₃ (77.23 ppm) for ¹³C NMR. ¹H NMR data were reported in order: chemical shift, multiplicity (s, singlet; d, doublet; t, triplet; q, quartet; m, multiplet), coupling constants (J, Hz), number of protons.

The Amber 11.0 molecular dynamics program package (D. A. Case et al. (2010), AMBER 11, University of California, San Francisco) was used to optimize the structure of the fullerene, the cage and the cage/fullerene binding complexes. The force field used was the general Amber force field (GAFF field) (Wang, J. et al. J. Comput. Chem. 2004, 25, 1157-1174) with the charge parameters computed by AM1-BCC method (Jakalian, A. et al. J. Comput. Chem. 2000, 21, 132-146). For each structure optimization run, the molecule was first minimized for 1000 steps using the conjugate gradient method, and then it was further optimized by simulated annealing method for 150 picosecond with a time-step of 1 femtosecond. During the simulated annealing, the system temperature was first raised up to 1000 K for 50 picosecond and then gradually cooled to 0 K for another 100 picosecond. Finally, the annealed structure was minimized again for another 1000 conjugate gradient steps and the final energy was recorded. The non-bonded interactions during the simulation were computed directly with a cutoff distance of 25 Å. A dielectric constant of 4.8 was assumed during the simulation, which is a typical value for organic solvents. By comparing the energies of the fullerene, the cage, and the binding complexes, the binding energy can be computed.

### Experimental Procedures

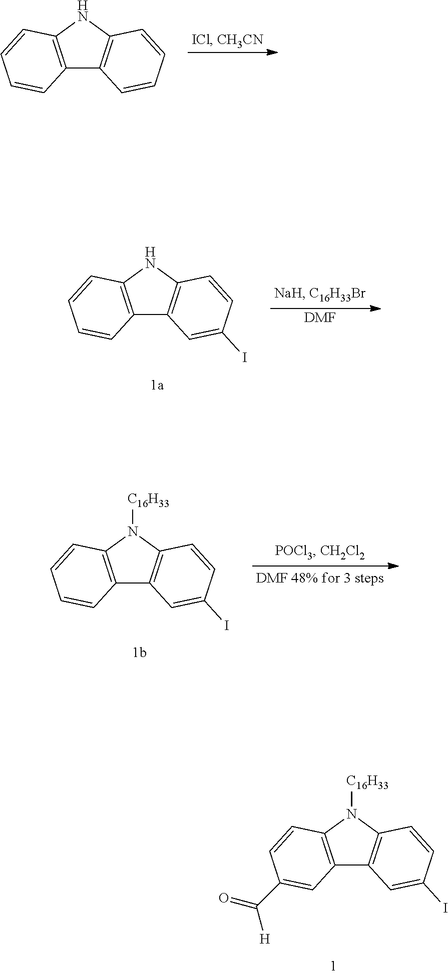

### 3-Formyl-N-hexadecyl-6-iodo-carbazole

To a solution of carbazole (5.00 g, 30.0 mmol) in CH₃CN (250 mL) was slowly added ICl (1.88 mL, 36 mmol) at 0° C. The mixture was stirred at 0° C. for 2 h, then slowly warmed up to room temperature and was stirred for another 2 h. The reaction was quenched with saturated aqueous Na₂SO₃ solution. The product was extracted with CH₂Cl₂ (80 mL×3). The organic extracts were combined and the volatiles were removed. The crude product of 3-iodocarbazole (1a) was collected as a white solid. (˜60% yield was determined by crude ¹H NMR spectra analysis.) Without further purification, the crude 3-iodocarbazole (1a) was dissolved in DMF (100 mL). NaH (1.80 g, 45 mmol, 60% dispersion in mineral oil) was added to the above solution and stirred for 5 mins at room temperature. Then 1-bromohexadecane (13.74 g, 45 mmol) was added. After stirring for 4 h at room temperature, the solvent was removed. 1 M HCl (100 mL) was added to the residue, and the mixture was extracted with CH₂Cl₂ (3×100 mL). The combined organic extracts were washed with water (100 mL), and brine (100 mL), dried over anhydrous Na₂SO₄, and concentrated to give the crude product. Purification by flash column chromatography (CH₂Cl₂:Hexane, 1:3 v/v) gave the product (1b) together with N-hexadecyl-3,6-diiodo-carbazole. To a mixture of DMF (47 mL, 600 mmol) and 1,2-dichloroethane (50 mL) was added POCl₃ (47.5 mL, 510 mmol) dropwise at 0° C. The mixture was warmed up to 35° C. and N-hexadecyl-3-iodo-carbazole (1b) was added. After heating at 90° C. for 24 h, the mixture was cooled to ambient temperature and poured into water (500 mL). The product was extracted with chloroform (150 mL×3). The combined organic extracts were washed with water (200 mL), and brine (200 mL), dried over anhydrous MgSO₄ and concentrated. The residue was purified via flash column chromatography (CH₂Cl₂:Hexane, 1:1 v/v) to provide pure product 1 as a white solid (7.85 g, 48% in three steps): ¹H NMR (500 MHz, CDCl₃): δ 10.08 (s, 1H), 8.51 (d, J=1.5 Hz, 1H), 8.44 (d, J=1.5 Hz, 1H), 8.03 (dd, J₁=8.5 Hz, J₂=1.5 Hz, 1H), 7.77 (dd, J₁=8.5 Hz, J₂=1.5 Hz, 1H), 7.46 (d, J=8.5 Hz, 1H), 7.22 (d, J=8.5 Hz, 1H), 4.29 (t, J=7.0 Hz, 2H), 1.85 (m, 2H), 1.39-1.21 (m, 26H), 0.89 (t, J=7.0 Hz, 3H); ¹³C NMR (100 MHz, CDCl₃): δ 191.71, 144.02, 140.47, 135.10, 129.76, 129.06, 127.59, 125.53, 124.55, 121.87, 111.57, 109.40, 83.09, 43.71, 32.13, 29.90, 29.89, 29.87, 29.83, 29.78, 29.73, 29.64, 29.57, 29.51, 29.06, 27.39, 22.91, 14.35; HRMS (m/z): [M+H]⁺ calcd. for C₂₉H₄₀INO, 546.2233. found, 546.2227.

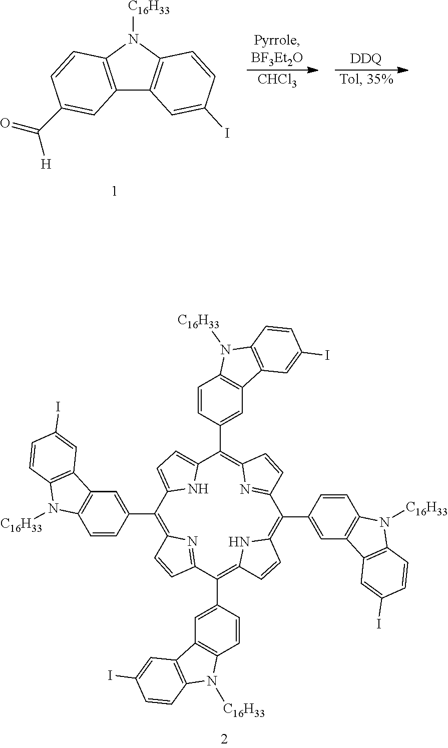

Compound 2:

To a solution of compound 1 (2.18 g, 4.0 mmol) and pyrrole (0.28 mL, 4.0 mmol) in chloroform (200 mL) was added BF₃.Et₂O (0.16 mL) dropwise at rt. The reaction mixture was stirred for 1 h at rt. A solution of 2,3-dichloro-5,6-dicyanobenzoquinone (0.68 g, 3.0 mmol) in toluene (10 mL) was added slowly. After stirring 1 h at rt, the reaction mixture was filtered through a silica gel pad. The volatiles were removed and the residue was purified by flash column chromatography (CH₂Cl₂:Hexane, 1:1 v/v) to provide the product 2 as a purple solid (0.826 g, 35%): ¹H NMR (500 MHz, CDCl₃): δ 8.95 (s, 4H), 8.91 (s, 8H), 8.54 (s, 4H), 8.41 (m, 4H), 7.81 (d, J=8.8 Hz, 4H), 7.73 (m, 4H), 7.34 (d, J=8.5 Hz, 4H), 4.47 (t, J=7.0 Hz, 8H), 2.07 (m, 8H), 1.53 (m, 8H), 1.45 (m, 8H), 1.39-1.23 (m, 88H), 0.90 (t, J=7.5 Hz, 12H), −2.41 (s, 2H); ¹³C NMR (100 MHz, CDCl₃): δ 140.61, 140.18, 134.33, 133.74, 133.63, 133.55, 131.50, 129.75, 126.96, 125.66, 120.90, 120.53, 111.20, 107.01, 81.72, 43.78, 32.13, 29.92, 29.87, 29.81, 29.70, 29.58, 29.36, 27.66, 22.91, 14.37; MALDI-TOF (m/z): [M+H]⁺ calcd. for C₁₃₂H₁₆₆I₄N₈, 2372.95. found: 2372.84.

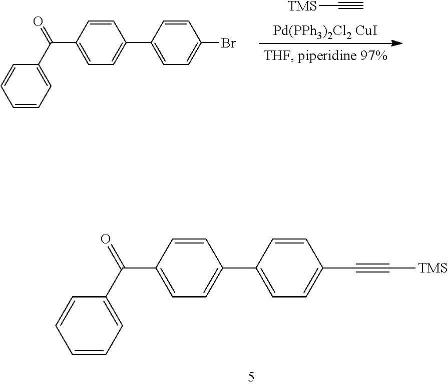

Compound 5:

The general Sonogashira's procedure was followed (Sonogashira, K. et al. Tetrahedron Letters 1975, 16, 4467-4470; Sonogashira, K. et al. Chem. Commun., 1977, 291-292). Using 4-benzoyl-4′-bromo-biphenyl (3.37 g, 10.0 mmol), trimethylsilylacetylene (1.47 g, 15.0 mmol), Pd(PPh₃)₂Cl₂ (0.210 g, 0.3 mmol), CuI (0.045 g, 0.25 mmol), piperidine (30 mL), and THF (30 mL), the product was obtained as a yellowish solid (3.45 g, 97%): ¹H NMR (500 MHz, CDCl₃): δ 7.89 (d, J=7.6 Hz, 2H), 7.83 (d, J=7.2 Hz, 2H), 7.69 (d, J=7.6 Hz, 2H), 7.59 (m, 5H), 7.50 (m, 2H), 0.29 (s, 9H); ¹³C NMR (100 MHz, CDCl₃): δ 196.3, 144.3, 139.9, 137.8, 136.6, 132.7, 132.6, 130.9, 130.1, 128.5, 127.2, 126.7, 123.2, 104.8, 95.8, 0.2; HRMS (m/z): [M+H]⁺ calcd. for C₂₄H₂₂OSi, 355.1518. found, 355.1518.

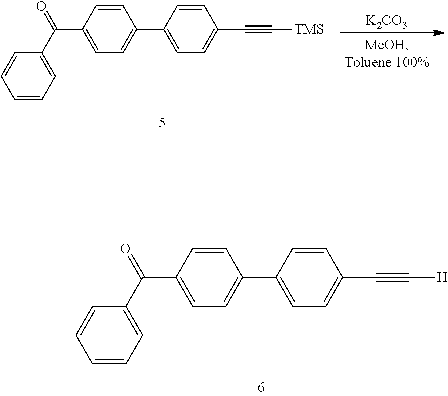

Compound 6:

To a solution of 5 (3.45 g, 9.7 mmol) in MeOH (50 mL) and toluene (50 mL) was added K₂CO₃ (2.68 g, 19.4 mmol). The mixture was stirred at room temperature for 1 h. The solvents were removed and the residue was dissolved in CH₂Cl₂ (100 mL). The organic solution was washed with saturated NH₄Cl (50 mL), and brine (50 mL), dried over Na₂SO₄, and concentrated. Purification by flash column chromatography (CH₂Cl₂:Hexane, 1:1 v/v) provided the product as a yellow solid (2.74 g 100%): ¹H NMR (500 MHz, CDCl₃): δ 7.91 (d, J=6.5 Hz, 2H), 7.85 (d, J=8.0 Hz, 2H), 7.71 (d, J=6.5 Hz, 2H), 7.61 (m, 5H), 7.52 (m, 2H), 3.18 (s, 1H); ¹³C NMR (100 MHz, CDCl₃): 6196.3, 144.2, 140.3, 137.7, 136.7, 132.8, 132.6, 130.9, 130.1, 128.5, 127.3, 127.0, 122.1, 83.4, 78.6; HRMS (m/z): [M+H]⁺ calcd. for C₂₁H₁₄O, 283.1123. found, 283.1125.

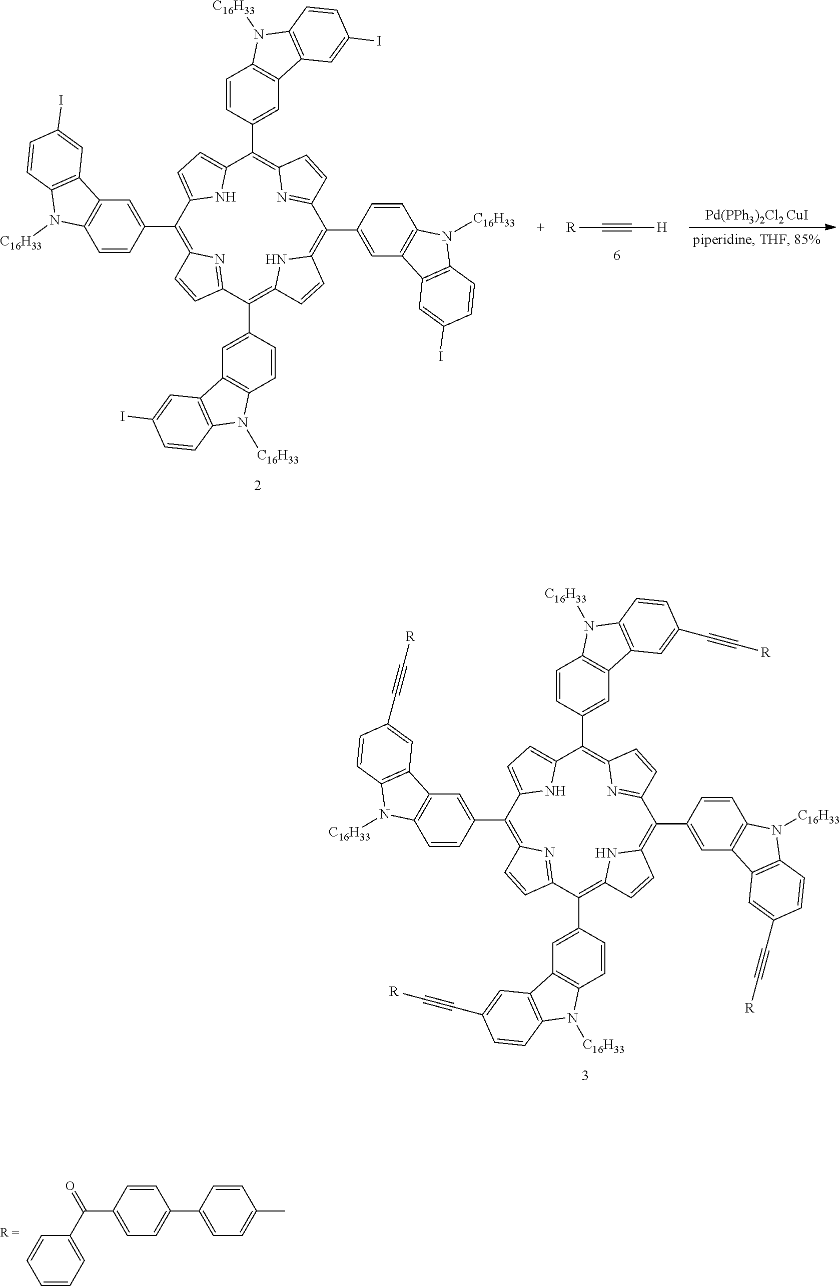

Compound 3:

The general Sonogashira's procedure was followed (Sonogashira, K. et al. Tetrahedron Letters 1975, 16, 4467-4470; Sonogashira, K. et al. Chem. Commun., 1977, 291-292). Compound 2 (500 mg, 0.21 mmol) was converted to monomer 3 using acetylene 6 (593 mg, 2.1 mmol), Pd(PPh₃)₂Cl₂ (23.6 mg, 0.034 mmol), CuI (4.2 mg, 0.022 mmol), piperidine (10 mL), and THF (50 mL). The product 3 was obtained as a purple solid (536 mg, 85%): ¹H NMR (500 MHz, CDCl₃): δ 9.01 (m, 4H), 8.94 (d, J=3.6 Hz, 8H), 8.46 (m, 8H), 7.90-7.45 (m, 64H), 4.56 (t, J=7.0 Hz, 8H), 2.11 (m, 8H), 1.59 (m, 8H), 1.48 (m, 8H), 1.41-1.22 (m, 88H), 0.86 (t, J=7.5 Hz, 12H), −2.39 (s, 2H); ¹³C NMR (100 MHz, CDCl₃, 59° C.*): δ 196.12, 144.58, 141.44, 140.86, 139.27, 138.16, 136.78, 134.11, 133.51, 132.44, 132.21, 131.54, 130.85, 130.15, 130.11, 129.93, 128.48, 127.27, 127.07, 126.90, 124.76, 124.32, 123.50, 121.67, 121.05, 114.01, 109.30, 107.16, 92.61, 87.82, 44.00, 32.12, 29.90, 29.88, 29.85, 29.71, 29.53, 29.46, 27.73, 22.86, 14.22; MALDI-TOF (m/z): [M+H]⁺ calcd. for C₂₁₆H₂₁₈N₈O₄, 2990.72. found, 2991.30.

The ¹³C NMR spectrum at room temperature shows multiple signals for several peaks (δ =196.12, 144.58, 138.16, 136.78, 130.85, 126.90), presumably due to the conformational inequivalence of the four ‘arms’ of 3.

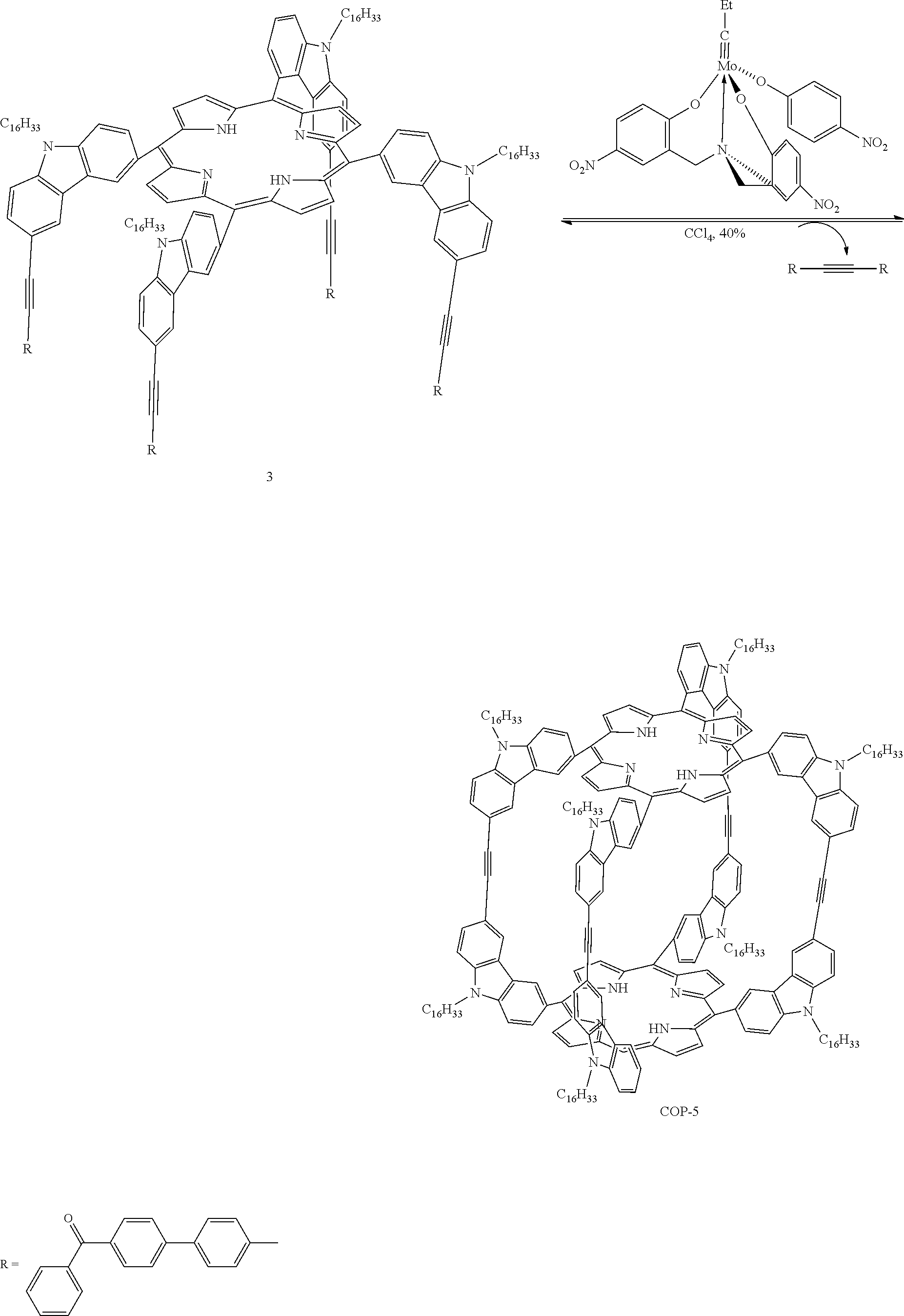

COP-5: The target cage compound was obtained by following the precipitation-driven alkyne metathesis procedures Jyothish, K. et al. Angew. Chem. Int. Ed. 2011, 50, 3435-3438; Moore, J. S.; Zhang, W. J. Am. Chem. Soc. 2004, 126, 12796-12796). The multidentate ligand (1.5 mg, 0.0032 mmol) and the Mo(VI) carbyne precursor (2.0 mg, 0.0031 mmol) were premixed in dry carbon tetrachloride (3 mL) for 20 minutes to generate the catalyst in situ. Subsequently, the monomer 3 (60 mg, 0.020 mmol) was added and the stirring was continued for 16 h at 60° C. under microwave irradiation. Another 3 mL fresh catalyst solution was prepared as described above and added, and the reaction mixture was stirred for another 16 h at 60° C., at which time the reaction was completed as monitored by GPC. Upon completion of the reaction, the reaction mixture was filtered to remove the byproduct and the filtrate was concentrated and subjected to flash column chromatography over alumina adsorption (CH₂Cl₂:Hexane, 1:1 v/v). The pure COP-5 was obtained as a purple solid (15 mg, 40%): ¹H NMR (400 MHz, CDCl₃): δ 8.77 (s, 8H), 8.64 (s, 16H), 8.27 (s, 8H), 8.21 (d, J=8.1 Hz, 8H), 7.74 (d, J=8.6 Hz, 8H), 7.62 (d, J=8.0 Hz, 8H), 7.50 (d, J=8.7 Hz, 8H), 4.49 (s, 16H), 2.11 (s, 16H), 1.65-1.15 (m, 208H), 0.88 (t, 7.0 Hz, 24H), −2.74 (s, 4H); ¹³C NMR (100 MHz, CDCl₃): δ 140.80, 140.58, 133.59, 131.81, 130.96, 129.36, 125.71, 124.33, 123.09, 120.98, 120.61, 114.51, 109.03, 106.38, 89.19, 43.84, 32.14, 29.92, 29.88, 29.73, 29.59, 29.48, 27.75, 22.92, 14.36; MALDI-TOF (m/z): [M+H]⁺ calcd. for C₂₇₂H₃₃₂N₁₆: 3825.66. Found: 3825.80.

Procedure for C₇₀ Purification:

To a mixture of C₇₀ (2.1 mg, 2.5 μmol) and C₆₀ (18 mg, 25 μmol, C₇₀/C₆₀, 1/10, 9 mol % for C₇₀) in CS₂ (5 mL) was added COP-5 (7.6 mg, 2.0 μmol). After sonication for 30 seconds, the solvent was evaporated and CHCl₃ (3 mL) was added. The undissolved solids were removed by centrifugation and the solution phase was treated with TFA (15 μL, 0.2 mmol). After sonication for 5 mins, dark solid precipitated, which were collected by centrifugation and washed with additional CHCl₃ (5 mL). The isolated fullerenes have a C₇₀/C₆₀ ratio of 3.4:1 (79 mol % for C₇₀), which was determined by UV-Vis absorption. The calculation is shown below:

Table 2
The absorbance of C₆₀, C₇₀ and the fullerene
mixture solution at 335 nm and 473 nm.

| | | |
|---|---|---|
| Fullerene | λ = 335 nm | λ = 473 nm |
| C₆₀ | 0.55189 | 0.00602 |
| C₇₀ | 0.29069 | 0.16063 |
| Mixture after | 0.35043 | 0.12620 |
| extraction | | |

The C₇₀/C₆₀ ratio in the fullerene mixtures were determined by the UV-Vis absorbance at 335 nm and 473 nm respectively. The standard solutions of C₆₀ (black), C₇₀ (red) were prepared with the concentrations of 8×10⁻⁶M in toluene. The UV-Vis absorption spectra were recorded for the standard C₆₀ and C₇₀ solutions with isosbestic point at 361 nm. The UV-Vis absorption of the fullerene mixture was measured and normalized to have the same isosbestic point (361 nm) with the above standard fullerene solutions. Given the absorbance of C₆₀, and C₇₀ standard solutions, the C₇₀/C₆₀ ratio in the fullerene mixture can be determined from the following equation.

$$\frac{{C}_{70}}{{C}_{60}} = \frac{{A}_{mix} - {A}_{C 60}}{{A}_{C 70} - {A}_{mix}}$$

The ratio of C₇₀/C₆₀ in the mixture after extraction that were calculated using the UV-Vis absorption at 335 nm and 473 nm are 3.37 and 3.49 respectively. Therefore, the C₇₀/C₆₀ ratio is estimated to be 3.4/1.

Procedure for the Synthesis of Macrocycle 1:

The tris(arylmethyl)amine ligand (1.5 mg, 0.0032 mmol) and the Mo(VI) carbyne precursor (2.0 mg, 0.0031 mmol) were premixed in dry carbon tetrachloride (3 mL) for 5 minutes to generate the catalyst in situ. Subsequently, the monomer 2 (77 mg, 0.040 mmol) was added and the reaction mixture was stirred at 45° C. for 16 hours. The reaction mixture was then filtered to remove the byproduct. The filtrate was concentrated and subjected to column chromatography over alumina (CH2C12/Hexane, 1/2, v/v). The pure product was obtained as a purple solid (33 mg, 60%).

1. A compound of the Formula A: 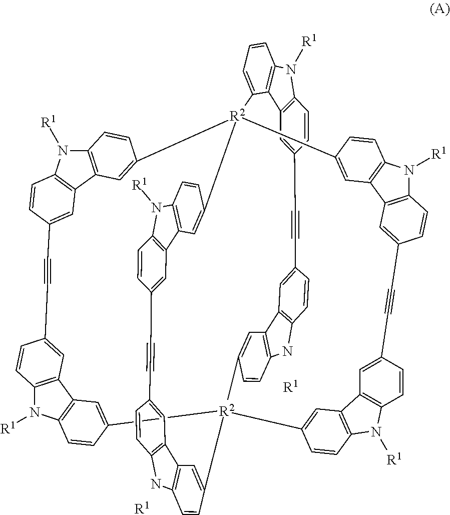
wherein
R¹ is a hydrophobic moiety or a hydrophilic moiety, and
R² is a monocyclic or fused hydrocarbon aromatic or heteroaromatic moiety, wherein the heteroaromatic moiety comprises one or more oxygen, nitrogen or phosphorous atoms.

2. A compound of the Formula B: 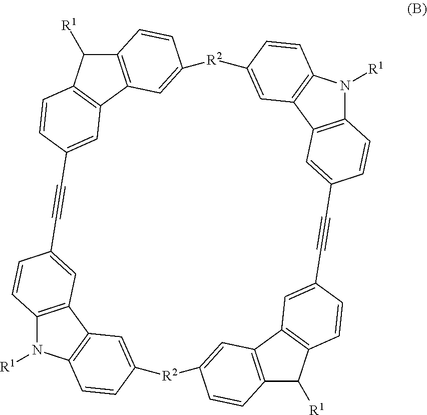
wherein
R¹ is a hydrophobic moiety or a hydrophilic moiety, and
R² is a monocyclic or fused hydrocarbon aromatic or heteroaromatic moiety, wherein the heteroaromatic moiety comprises one or more oxygen, nitrogen or phosphorous atoms, wherein R² is optionally further substituted with an aromatic group, and wherein the aromatic group is optionally further substituted with a C₁-C₆-alkyl.

3. The compound of claim 1 or 2, wherein R¹ is C₁-C₃₀-alkyl.

4. The compound of claim 1 or 2, wherein R¹ is PEG.

5. The compound of claim 1 or 2, wherein R² is pyrene, porphyrin, or phthalocyanine.

6. The compound of claim 1 or 2, wherein R² is porphyrin.

7. The compound of claim 2, wherein R² is optionally further substituted with a phenyl group, and wherein the phenyl group is optionally further substituted with a C₁-C₆-alkyl.

8. The compound of claim 1, wherein the compound of Formula A is a compound having the Formula I: 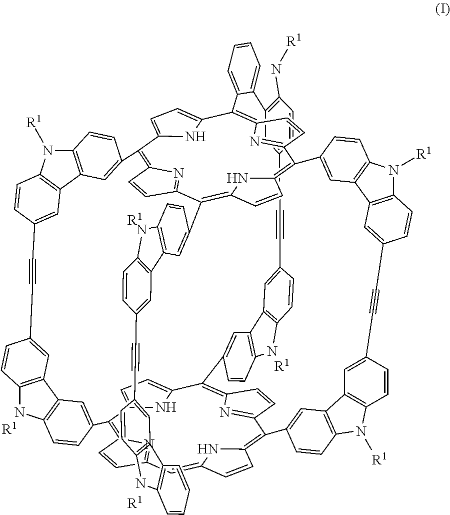
wherein R¹ is C₁-C₃₀-alkyl.

9. The compound of claim 2, wherein the compound of Formula B is a compound having the Formula II: 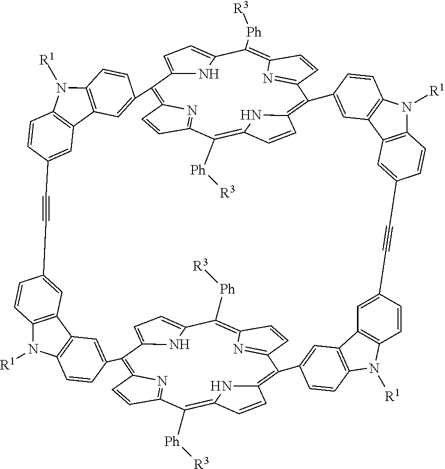
(II)
wherein R¹ is C₁-C₃₀-alkyl and R³ is C₁-C₆-alkyl.

10. The compound of claim 8 or 9, wherein R¹ is C₁₀-C₂₀-alkyl.

11. The compound of claim 10, wherein R¹ is C₁₆H₃₃.

12. The compound of claim 9, wherein R³ is t-butyl.

13. A method of preparing a compound of Formula I as specified in claim 8, comprising reacting a compound of Formula 3: 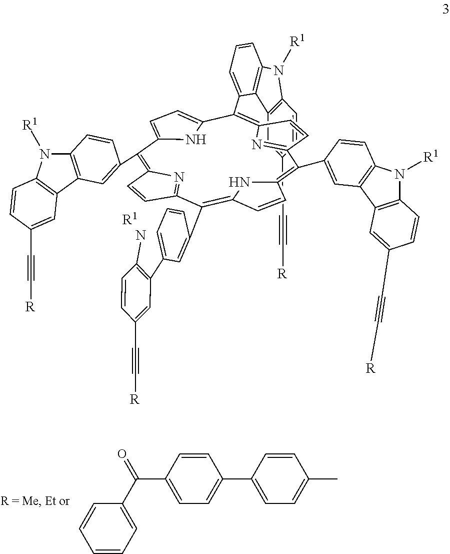
with a compound of formula 4: 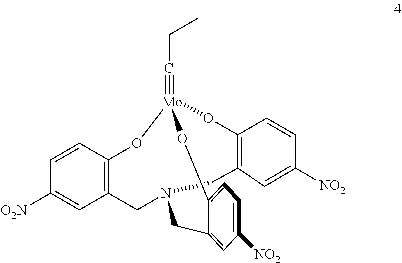
such that the compound of Formula I is produced, wherein R¹ is C₁-C₃₀-alkyl.

14. A method of preparing a compound of Formula II as specified in claim 9, comprising reacting a compound of Formula 5: 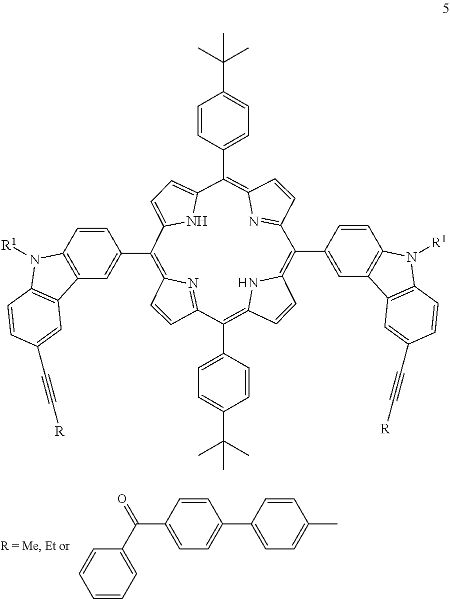
with a compound of formula 4: 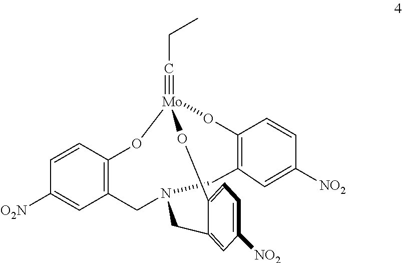
such that the compound of Formula II is produced, wherein R¹ is C₁-C₃₀-alkyl.

15. A method for separating fullerenes from a mixture comprising fullerenes, the method comprising contacting the mixture with a compound of Formula I: 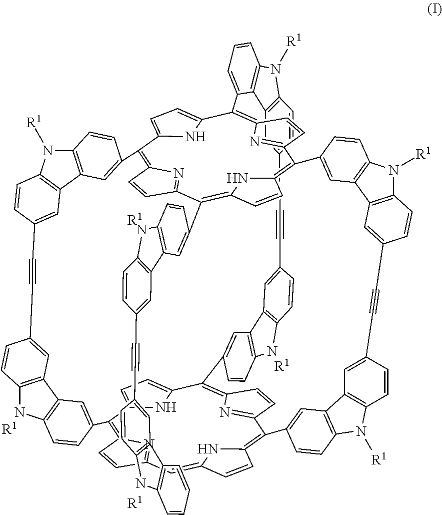
wherein R¹ is C₁-C₃₀-alkyl;
to generate a Formula I-fullerene complex.

16. A method for separating fullerenes from a mixture comprising fullerenes, the method comprising contacting the mixture with a compound of Formula II: 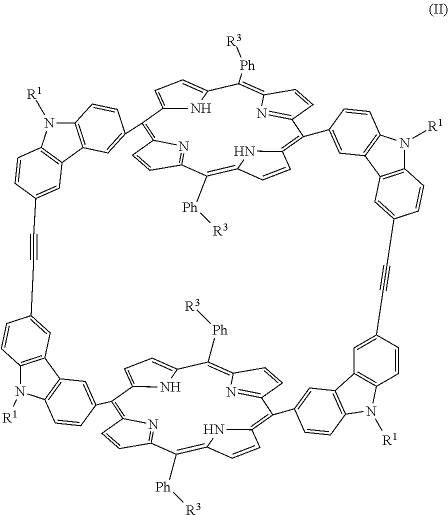
wherein R¹ is C₁-C₃₀-alkyl and R³ is C₁-C₆-alkyl.

17. The method of claim 15 or 16, further comprising removing the Formula I-fullerene complex or the Formula II-fullerene complex from the mixture.

18. The method of claim 17, further comprising separating the fullerene from the Formula I-fullerene complex or the Formula II-fullerene complex.

19. The method of claim 18, wherein the fullerene is separated from the Formula I-fullerene complex or Formula II-fullerene complex by contacting the complex with acid.

20. The method of claim 19, wherein the acid is trifluroacetic acid.

21. The method of any one of claims 15-20, wherein the fullerene to be extracted is C₆₀, C₇₀, or a mixture thereof.

22. The method of any one of claims 15-20, wherein the fullerene to be extracted is C₈₄.

23. The method of any one of claims 15-20, wherein the mixture containing fullerenes comprises C₆₀, C₇₀, C₇₆, or C₈₄, or other higher or lower molecular weight fullerenes represented by C₂₀₊₂ₘ where m is an integer.

24. A method for separating C₇₀ fullerenes from a mixture comprising C₆₀ and C₇₀ fullerenes, wherein the method comprises contacting the mixture with a compound of Formula I: 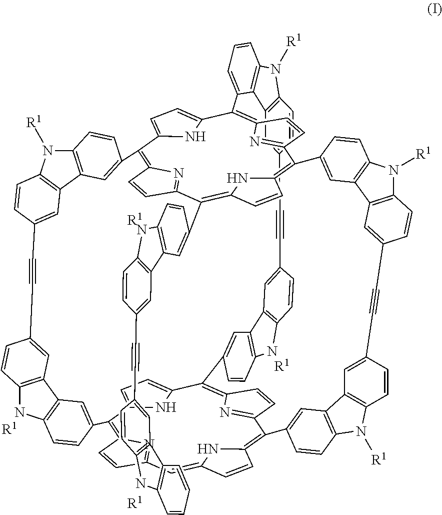
wherein R¹ is C₁-C₃₀-alkyl;
to generate a Formula I-C₇₀ complex.

25. The method of claim 24, further comprising removing the Formula I-C₇₀ complex from the mixture.

26. The method of claim 25, further comprising separating the C₇₀-fullerene from the Formula I-C₇₀ complex.

27. The method of claim 26, wherein the C₇₀-fullerene is separated from the Formula I-C₇₀ complex by contacting the complex with acid.

28. The method of claim 27, wherein the acid is trifluroacetic acid.

29. A method for separating C₈₄ fullerenes from a mixture comprising C₈₄ fullerenes and at least one of C₆₀ or C₇₀ fullerenes, wherein the method comprises contacting the mixture with a compound of Formula II: 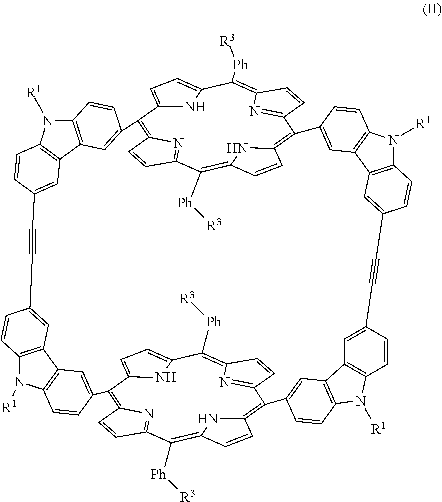
wherein R¹ is C₁-C₃₀-alkyl and R³ is C₁-C₆-alkyl.

30. The method of claim 15, 16, 24 or 29, wherein the separation takes place in a solvent.

31. The method of claim 30, wherein the solvent is tetrahydrofuran, dioxane, toluene, or dichloromethane.

32. A complex comprising a compound of Formula I as specified in claim 8 and C₇₀ fullerene.

33. A complex comprising a compound of Formula I as specified in claim 8 and C₆₀ fullerene.

34. The complexes of claims 19 or 20 wherein R¹ of Formula I is C₁₀-C₂₀-alkyl.

35. The complexes of claim 19 or 20 wherein R¹ of Formula I is C₁₆H₃₃.

36. A complex comprising a compound of Formula II as specified in claim 9 and C₈₄ fullerene.

37. A molecular cage prepared from a single monomer, comprising the same top and bottom molecular structures, wherein the top and bottom molecules are linked through an ethynylene group to form a non-collapsible structure.

38. The molecular cage of claim 23, wherein the top and bottom molecules are porphyrin or phthalocyanine.

39. The molecular cage of claim 24, wherein the porphyrin or phthalocyanine groups are substituted with carbazole.
